(19) United States
(12) Patent Application Publication
(10) Pub. No.: US 2024/0350487 A1
(43) Pub. Date: Oct. 24, 2024

(54) METHOD OF DOSING VAPENDAVIR TO TREAT ENTEROVIRUS INFECTIONS

(71) Applicant: Altesa BioSciences, Inc., College Park, GA (US)

(72) Inventors: Brett Paul GIROIR, College Station, TX (US); John Michael CLERICI, Reston, VA (US); Seth Allen RUDNICK, Pinehurst, NC (US); Katie LAESSIG, Washington, DC (US); Katherine Elizabeth SQUIRES, Atlanta, GA (US); Andrea True KELLY, Charleston, SC (US)

(73) Assignee: Altesa BioSciences, Inc., College Park, GA (US)

(21) Appl. No.: 18/604,264

(22) Filed: Mar. 13, 2024

Related U.S. Application Data
(60) Provisional application No. 63/489,929, filed on Mar. 13, 2023.
(60) Provisional application No. 63/489,936, filed on Mar. 13, 2023.
(60) Provisional application No. 63/578,073, filed on Aug. 22, 2023.
(60) Provisional application No. 63/578,078, filed on Aug. 22, 2023.

(51) Int. Cl. A61K 31/501; A61P 31/16

(52) U.S. Cl. CPC A61K 31/501; A61P 31/16

(57) ABSTRACT

The present disclosure describes methods of dosing vapendavir or a pharmaceutically acceptable salt thereof to treat subject in need thereof, thereby treating the respiratory enterovirus infection, wherein the respiratory enterovirus infection may be a respiratory enterovirus infection. The subject may further have an underlying condition, and the methods of the present disclosure may be useful in preventing or reducing acute exacerbations of the underlying condition in the subject. Pharmaceutical formulations of vapendavir are also disclosed.

### CROSS REFERENCE TO RELATED APPLICATIONS

This application claims the benefit of priority under 35 U.S.C. § 119 (e) of U.S. Provisional Patent Application Ser. No. 63/489,929 filed on Mar. 13, 2023, U.S. Provisional Patent Application Ser. No. 63/578,073 filed on Aug. 28, 2023, U.S. Provisional Patent Application Ser. No. 63/489,936 filed on Mar. 13, 2023, and U.S. Provisional Patent Application Ser. No. 63/578,078 filed on Aug. 28, 2023. The disclosures of the prior applications are considered part of and are herein incorporated by reference in the disclosure of this application in their entirety.

### BACKGROUND

Rhinoviruses (RVs) are small RNA viruses that represent significant diversity within the Enterovirus (EV) genus, with over 180 distinct strains. RVs are the most common cause of upper respiratory tract infections (UTRI) and a frequent cause of community-acquired pneumonia in adults. While RVs are normally associated with mild and self-limiting cold-like symptoms. RVs can cause significant morbidity and mortality via the exacerbation of chronic respiratory diseases such as asthma, cystic fibrosis, primary ciliary dyskinesia, sickle cell disease, cancer, or chronic obstructive pulmonary disease (COPD).

Acute exacerbations are the primary contributors to the morbidity and mortality associated with chronic respiratory diseases. These exacerbations are associated with an impaired quality of life and accelerated deterioration of lung function. For example, approximately 40-50% of COPD exacerbations are linked to RV infection. However, current therapies for managing chronic respiratory diseases and treating acute exacerbations are aimed at palliating the symptoms and, in many cases, have significant side effects. In the case of COPD, current therapies such as long-acting muscarinic receptor antagonists (LAMAs), long acting β2 receptor agonists (LABAs), inhaled corticosteroids (ICS), and antibacterial drugs are only partially effective and fail to address the underlying mechanisms of disease or triggers of exacerbation.

Vapendavir (VPV) is a potent, broad spectrum antiviral drug active against a majority of RV serotypes and other enteroviruses evaluated in cell-based assay systems and human clinical trials. Vapendavir has been tested in humans with no appreciable safety concerns noted following single and multiple doses. There remains a need for an effective treatment for RV infections, particularly in subjects having chronic respiratory conditions such as asthma, cystic fibrosis, primary ciliary dyskinesia, sickle cell disease, cancer, or COPD.

### SUMMARY

Embodiments herein are directed to methods of treating a respiratory enterovirus infection in a human subject in need thereof, including orally administering to the subject a therapeutically effective amount of vapendavir or a pharmaceutically acceptable salt thereof, thereby treating the respiratory enterovirus infection in the subject.

In some embodiments, the respiratory enterovirus infection is selected from a rhinovirus, echovirus, EV-68, EV-71, coxsackie virus, a non-polio enterovirus and combinations thereof.

In some embodiments, the therapeutically effective amount of vapendavir or a pharmaceutically acceptable salt thereof is between about 250 mg and about 2,000 mg per day.

In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days.

In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses.

In some embodiments, the therapeutically effective amount of vapendavir or a pharmaceutically acceptable salt thereof is an amount that achieves a Cₘₐₓ of about 2000 to about 12000 ng/ml.

In some embodiments, the single loading dose is administered within about 48 hours of the subject displaying symptoms of a respiratory enterovirus infection.

In some embodiments, the subject is asymptomatic but has been diagnosed with a respiratory enterovirus infection.

In some embodiments, the at least one maintenance dose is first administered about 6 to about 12 hours following administering the single loading dose.

In some embodiments, the single loading dose is administered after the subject has been diagnosed with a respiratory enterovirus infection, wherein the diagnosis is achieved by a clinical diagnosis, multiplex PCR testing, rapid antigen testing, direct fluorescent antibody testing, arterial blood gas testing, chest x-rays, peak flow meter testing, pleural fluid cultures, lung CT scanning, lung function tests, pulse oximetry testing, spirometry testing, sputum testing, nasal swab testing, throat swab testing, or any combination thereof.

In some embodiments, the subject is in a fed state at the time of administration.

In some embodiments, the subject is a human. In some embodiments, the subject is a human between about 40 and about 85 years of age. In some embodiments, the subject is a human under the age of 6 years of age.

In some embodiments, the single loading dose and maintenance dose are administered within about 30 minutes of the subject consuming a meal with solid food. In some embodiments, the single loading dose and maintenance dose are administered within about 30 minutes of the subject consuming a high fat meal with solid food. In some embodiments, administering the single loading dose and maintenance dose to a subject within about 30 minutes of the subject consuming a meal with solid food results in a higher plasma concentration of vapendavir or a pharmaceutically acceptable salt thereof than administering the single loading dose and maintenance dose to a subject in a fasted state.

In some embodiments, the subject has an underlying condition selected from chronic obstructive pulmonary diseases (COPD), asthma, mixed COPD and bronchitis; pulmonary hypertension: pulmonary edema: acute respiratory distress syndrome (ARDS); pneumoconiosis: interstitial lung disease: mesothelioma: sickle cell disease, cancer, or any combination thereof. In some embodiments, the subject has COPD and has been diagnosed with GOLD stage 1 COPD, GOLD stage 2 COPD, GOLD stage 3 COPD, or GOLD stage 4 COPD.

In some embodiments, the subject is receiving stable maintenance therapy for COPD.

In some embodiments, treating the respiratory enterovirus infection in the subject includes decreasing a viral load in the sputum, nasal passages, or a combination thereof in the subject compared with a viral load in the sputum, nasal passages, or a combination thereof of the subject prior to treatment.

In some embodiments, treating the respiratory enterovirus infection in the subject includes decreasing, or preventing an increase in the subjects' Evaluating Respiratory Symptoms in COPD Score (E-RS) compared to the subjects' Evaluating Respiratory Symptoms in COPD Score (E-RS) prior to treatment.

In some embodiments, treating the respiratory enterovirus infection in the subject includes decreasing, preventing an increase in the subjects' peak lower respiratory symptom score (LRSS) compared to the subjects' peak lower respiratory symptom score (LRSS) prior to treatment.

In some embodiments, treating the respiratory enterovirus infection in the subject includes decreasing, or preventing an increase in the subjects' peak upper respiratory symptom score (URSS) compared to the subjects' peak upper respiratory symptom score (URSS) prior to treatment.

In some embodiments, treating the respiratory enterovirus infection in the subject includes decreasing, or preventing an increase in the subjects' EXAcerbation of Chronic Pulmonary Disease Tool-Patient Reported Outcome (EXACT-PRO) compared to the subjects' EXAcerbation of Chronic Pulmonary Disease Tool-Patient Reported Outcome (EXACT-PRO) prior to treatment.

In some embodiments, treating the respiratory enterovirus infection in the subject includes decreasing, or preventing an increase in the subjects' EXAcerbation of Chronic Pulmonary Disease Tool-Patient Respiratory Symptoms (EXACT-RS) compared to the subjects' EXAcerbation of Chronic Pulmonary Disease Tool-Patient Respiratory Symptoms (EXACT-RS) prior to treatment.

In some embodiments, treating the respiratory enterovirus infection in the subject includes decreasing, or preventing an increase in the subjects' COPD assessment tool (CAT) compared to the subjects' COPD assessment tool (CAT) prior to treatment.

In some embodiments, treating the respiratory enterovirus infection in the subject includes decreasing, or preventing an increase in the subjects' St. George's Respiratory Questionnaire (SGRQ) compared to the subjects' St. George's Respiratory Questionnaire (SGRQ) prior to treatment.

In some embodiments, treating the respiratory enterovirus infection in the subject includes decreasing, or preventing an increase in the subjects' Wisconsin Upper Respiratory Symptom Survey (WURSS) compared to the subjects' Wisconsin Upper Respiratory Symptom Survey (WURSS) prior to treatment.

In some embodiments, treating the respiratory enterovirus infection in the subject includes improving the subject's lung function compared to the subjects' lung function prior to treatment.

In some embodiments, the subjects' lung function is measured by Forced Expiratory Volume-1 (FEV₋₁: in liters and % predicted), Forced Vital Capacity (FVC; in liters and % predicted) FEV₁/FVC ratio and peak expiratory flow (PEF), or any combination thereof.

In some embodiments, treating the respiratory enterovirus infection in the subject includes improving, or preventing a decrease in the subject's lung function compared to the subjects' lung function prior to treatment.

In some embodiments, the subjects' lung function is measured by Forced Expiratory Volume-1 (FEV₋₁: in liters and % predicted), Forced Vital Capacity (FVC: in liters and % predicted) FEV 1/FVC ratio and peak expiratory flow (PEF), or any combination thereof.

In some embodiments, treating the respiratory enterovirus infection in the subject includes reducing, or preventing an increase in the subjects' peak nasal lavage viral load compared to the subjects' peak nasal lavage viral load prior to treatment.

In some embodiments, treating the respiratory enterovirus infection in the subject includes reducing, or preventing an increase in the subjects' peak sputum lavage viral load compared to the subjects' peak sputum lavage viral load prior to treatment.

In some embodiments, treating the respiratory enterovirus infection in the subject includes reducing, or preventing an increase in the subjects' AUC nasal viral load compared to the subjects' AUC nasal viral load prior to treatment.

In some embodiments, treating the respiratory enterovirus infection in the subject includes reducing, or preventing an increase in the subjects' AUC sputum viral load compared to the subjects' AUC sputum viral load prior to treatment.

In some embodiments, treating the respiratory enterovirus infection in the subject includes reducing the duration of viral shedding.

In some embodiments, treating the respiratory enterovirus infection in the subject includes reducing, or preventing an increase in the subjects' peak sputum bacterial load compared to the subjects' peak sputum lavage bacterial load prior to treatment.

In some embodiments, treating the respiratory enterovirus infection in the subject includes reducing, or preventing an increase in the subjects' AUC sputum bacterial load compared to the subjects' AUC sputum bacterial load prior to treatment.

In some embodiments, treating the respiratory enterovirus infection in the subject includes reducing the number of days wherein the subject is positive for bacteria in the sputum.

In some embodiments, treating the respiratory enterovirus infection in the subject includes preventing, or reducing, acute exacerbations of the underlying condition in the subject caused by the respiratory enterovirus infection.

In some embodiments, treating the respiratory enterovirus infection in the subject includes a reduction in the frequency of acute exacerbations of the underlying condition in the subject, a reduction in the severity of acute exacerbations of the underlying condition in the subject, a reduction in the duration of acute exacerbations of the underlying condition in the subject or combinations thereof, caused by the respiratory enterovirus infection.

In some embodiments, treating the respiratory enterovirus infection in the subject includes preventing an increase in the frequency of acute exacerbations of the underlying condition in the subject, an increase in the severity of acute exacerbations of the underlying condition in the subject, an increase in the duration of acute exacerbations of the underlying condition in the subject or combinations thereof, caused by the respiratory enterovirus infection.

In some embodiments, administering to the subject a therapeutically effective amount of vapendavir or a pharmaceutically acceptable salt thereof exceeds the paEC₅₀ for human rhinoviruses. In some embodiments, the paEC₅₀ for human rhinoviruses is about 75.4 ng/ml.

In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₅₀ for human rhinoviruses is exceeded for about 4 to about 10 days or until treatment is stopped.

In some embodiments, administering to the subject a therapeutically effective amount of vapendavir or a pharmaceutically acceptable salt thereof exceeds the paEC₉₀ for human rhinoviruses. In some embodiments, the paEC₉₀ for human rhinoviruses is about 678.6 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₉₀ for human rhinoviruses is exceeded for about 4 to about 10 days or until treatment is stopped.

In some embodiments, administering to the subject a therapeutically effective amount of vapendavir or a pharmaceutically acceptable salt thereof results in a Cₘₐₓ that exceeds the paEC₅₀ for human rhinoviruses. In some embodiments, the paEC₅₀ for human rhinoviruses is about 75.4 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₅₀ for human rhinoviruses is exceeded for about 4 to about 10 days or until treatment is stopped.

In some embodiments, administering to the subject a therapeutically effective amount of vapendavir or a pharmaceutically acceptable salt thereof results in a Cₘₐₓ that exceeds the paEC₉₀ for human rhinoviruses. In some embodiments, the paEC₉₀ for human rhinoviruses is about 678.6 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₉₀ for human rhinoviruses is exceeded for about 4 to about 10 days or until treatment is stopped.

In some embodiments, administering to the subject a therapeutically effective amount of vapendavir or a pharmaceutically acceptable salt thereof exceeds the paEC₅₀ for human rhinoviruses following a single dose. In some embodiments, the paEC₅₀ for human rhinoviruses is about 75.4 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₅₀ for human rhinoviruses is exceeded for about 4 to about 10 days or until treatment is stopped.

In some embodiments, administering to the subject a therapeutically effective amount of vapendavir or a pharmaceutically acceptable salt thereof exceeds the paEC₉₀ for human rhinoviruses. In some embodiments, the paEC₉₀ for human rhinoviruses is about 678.6 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₉₀ for human rhinoviruses is exceeded for about 4 to about 10 days or until treatment is stopped.

In some embodiments, the therapeutically effective amount of vapendavir or a pharmaceutically acceptable salt thereof is in tablet form. In some embodiments, the vapendavir is a free base, maleate salt, oxalate salt, phosphate salt, sulfate salt, tartrate salt, or chloride salt. In some embodiments, the vapendavir or a pharmaceutically acceptable salt thereof is an amorphous form of vapendavir.

Some embodiments are directed to pharmaceutical compositions including a therapeutically effective amount of vapendavir or a pharmaceutically acceptable salt thereof. In some embodiments, the vapendavir is a free base, or a pharmaceutically acceptable salt thereof, such as a maleate salt, oxalate salt, phosphate salt, sulfate salt, tartrate salt or chloride salt. In some embodiments, the vapendavir is an amorphous form of vapendavir or a pharmaceutically acceptable salt thereof. In some embodiments, the pharmaceutical composition includes about 250 mg of vapendavir or a pharmaceutically acceptable salt thereof. In some embodiments, the pharmaceutical composition is a tablet.

### DRAWINGS

Aspects, features, benefits, and advantages of the embodiments described herein will be apparent with regard to the following description, appended claims, and accompanying drawings where:

FIG. 1 is a diagram of the study design in a clinical study of vapendavir for the treatment of respiratory enterovirus infections in subjects with COPD.

FIG. 2 depicts mean vapendavir plasma concentrations vs time profiles (Top=Linear Scale, Bottom=Log Scale) for normal healthy volunteers (Group A) (Periods 1 and 2 (Fast/Fed).

FIG. 3 depicts mean vapendavir plasma concentrations vs time profiles (Top=Linear Scale, Bottom=Log Scale) normal healthy volunteers (Group A) (Treatment C) (Days 1-7). Day 1=VPV 1000 mg am and 500 mg pm: Day 4=VPV 500 mg BID: Day 7=VPV 500 mg BID.

FIG. 4 depicts mean vapendavir plasma concentrations vs time profiles (Top=Linear Scale, Bottom=Log Scale) for participants with a diagnosis of COPD (Group B)—Treatment C (Days 1-7). SD=Standard deviation Day 1=VPV 1000 mg am and 500 mg pm; Day 4=VPV 500 mg BID: Day 7=VPV 500 mg BID.

FIG. 5 depicts the mean plasma concentration following administration of VPV for Group B—Treatment C (Days 1-7 (Day 1=VPV 1000 mg am and 500 mg pm: Day 4=VPV 500 mg BID: Day 7=VPV 500 mg BID)) and shows the average protein adjusted EC₅₀ for human rhinoviruses.

FIG. 6 depicts the mean plasma concentration in participants with COPD dosed with a 1,000 mg loading dose followed by 500 mg BID for a full 7-day course.

FIG. 7 depicts the mean Cₘₐₓ in fasted versus fed participants with COPD after 1, 4 and 7 days for the fasted group (Group B—Treatment C (Days 1-7 (Day 1=VPV 1000 mg am and 500 mg pm: Day 4=VPV 500 mg BID: Day 7=VPV 500 mg BID)) and 1, 2, and 7 day's for the fed group (1000 mg loading dose followed by 500 mg BID for a full 7-day course).

FIG. 8 depicts the mean Cₘₐₓ in fasted versus fed participants with COPD as well as normal healthy volunteers after 1, 4 and 7 days for the fasted group (Group A (normal healthy volunteers) and Group B (COPD)—Treatment C (Days 1-7 (Day 1=VPV 1000 mg am and 500 mg pm: Day 4=VPV 500 mg BID: Day 7=VPV 500 mg BID)) and 1, 2, and 7 day's for the fed group (1000 mg loading dose followed by 500 mg BID for a full 7-day course).

FIG. 9 depicts the mean Cₘᵢₙ (12 hours) in fasted versus fed participants with COPD after 1, 4 and 7 days for the fasted group (Group B—Treatment C (Days 1-7 (Day 1=VPV 1000 mg am and 500 mg pm: Day 4=VPV 500 mg BID: Day 7=VPV 500 mg BID))) and 1, 2, and 7 days for the fed group (1000 mg loading dose followed by 500 mg BID for a full 7-day course).

FIG. 10 depicts the mean Cₘᵢₙ (12 hours) in fasted versus fed participants with COPD as well as normal healthy volunteers after 1, 4 and 7 days for the fasted group (Group A (normal healthy volunteers) and Group B (COPD)—Treatment C (Days 1-7 (Day 1=VPV 1000 mg am and 500 mg pm: Day 4=VPV 500 mg BID: Day 7=VPV 500 mg BID)) and 1, 2, and 7 days for the fed group (1000 mg loading dose followed by 500 mg BID for a full 7-day course).

FIG. 11 depicts Cₘₐₓ for each day of treatment for each of the COPD subjects in the study where each subject was fed.

FIG. 12 depicts Cₘₐₓ for each day of treatment for each of the COPD subjects in the study where each subject was fasted (Group B).

FIG. 13. depicts the coefficient of variation for the Cₘₐₓ in fasted versus fed participants with COPD after 1, 4 and 7 days for the fasted group (Group B—Treatment C (Days 1-7 (Day 1=VPV 1000 mg am and 500 mg pm: Day 4=VPV 500 mg BID: Day 7=VPV 500 mg BID))) and 1, 2, and 7 days for the fed group (1000 mg loading dose followed by 500 mg BID for a full 7-day course).

FIG. 14. depicts the coefficient of variation for the Cₘᵢₙ in fasted versus fed participants with COPD after 1, 4 and 7 days for the fasted group (Group B—Treatment C (Days 1-7 (Day 1=VPV 1000 mg am and 500 mg pm: Day 4=VPV 500 mg BID: Day 7=VPV 500 mg BID))) and 1, 2, and 7 days for the fed group (1000 mg loading dose followed by 500 mg BID for a full 7-day course).

FIG. 15 depicts Kₑₗ (Elimination Rate Constant) for each of the 6 Participants for the fed group (1000 mg loading dose followed by 500 mg BID for a full 7-day course) at day 7.

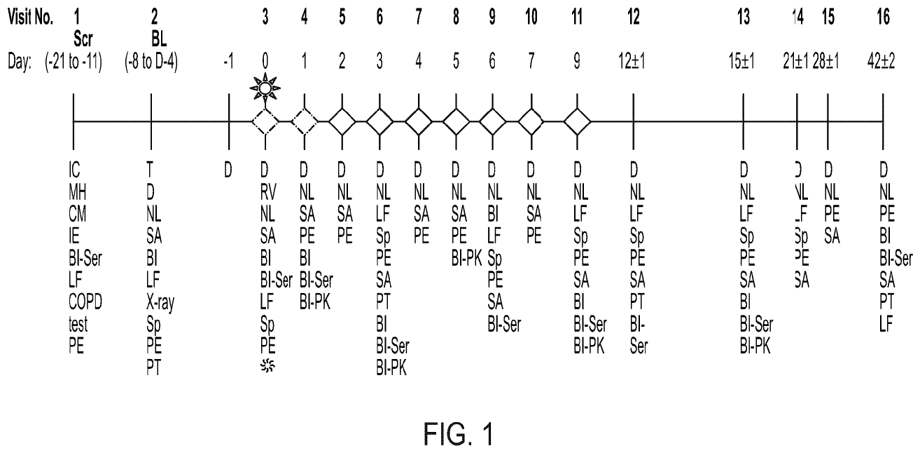

### DETAILED DESCRIPTION

Before compounds, compositions and methods are described in detail, it is to be understood that this disclosure is not limited to the particular processes, compositions, or methodologies described, as these may vary. It is also to be understood that the terminology used in the description is for the purpose of describing the particular versions or embodiments only, and is not intended to limit the scope of the disclosure which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the disclosure, the preferred methods, devices, and materials are now described.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein, the term “about” means plus or minus up to 20% of the numerical value of the number with which it is being used. For example, “about 50%” means in the range of 40-60% and includes exactly 50%. The term “about” may refer to plus or minus 1%, 5%, 10%, 15%, or 20% of the numerical value of the number with which it is being used.

“Administering,” when used in conjunction with the compounds of the disclosure, means to administer a compound directly into or onto a target tissue or to administer a compound systemically or locally to a patient or other subject.

The term “animal” as used herein includes, but is not limited to, humans and non-human vertebrates such as wild, experimental, domestic, and farm animals, and pets. As used herein, the terms “subject,” “individual,” and “patient,” are used interchangeably and refer to any animal, including mammals, mice, rats, other rodents, rabbits, dogs, cats, swine, cattle, sheep, horses, primates, non-human primates, humans, and the like.

The term “improves” is used to convey that the disclosure changes either the characteristics and/or the physical attributes of the tissue to which it is being provided, applied or administered. The term “improves” may also be used in conjunction with a disease state such that when a disease state is “improved” the symptoms or physical characteristics associated with the disease state are diminished, reduced, eliminated, delayed, or averted.

The term “inhibiting” includes the blockade, aversion of a certain result or process, or the restoration of the converse result or process. In terms of treatment by administration of a compound of the disclosure, “inhibiting” includes protecting against (partially or wholly) or delaying the onset of symptoms, alleviating symptoms, or protecting against, diminishing or eliminating a disease, condition, or disorder.

As used herein, the term “free base” refers to a non-salt form of a compound as described herein.

The term “salts” can include acid addition salts or addition salts of free bases. The salts as described herein may be pharmaceutically acceptable. Examples of acids which may be employed to form pharmaceutically acceptable acid addition salts include but are not limited to salts derived from nontoxic inorganic acids such as nitric, phosphoric, sulfuric, hydrobromic, hydroiodic, hydrofluoric, or phosphorus acids, as well as salts derived from nontoxic organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, hydrochloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Salts of amino acids are also contemplated, such as arginate, gluconate, galacturonate, and the like.

Those skilled in the art of organic chemistry will appreciate that many organic compounds can form complexes with solvents in which they are reacted or from which they are precipitated or crystallized. For example, a complex with water is known as a “hydrate.” Solvates of the compounds of the present disclosure are within the scope of this disclosure. The salts of the compound of any of the formulae described herein may form solvates (e.g., hydrates) and the present disclosure includes all such solvates. The meaning of the word “solvates” is well known to those skilled in the art as a compound formed by interaction of a solvent and a solute (i.e., solvation). Techniques for the preparation of solvates are well established in the art.

As used herein, unless specifically indicated, the term “active ingredient” refers to a compound of any of the formulae as described herein.

The phrase “pharmaceutically acceptable” refers to molecular entities and compositions that are generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a subject. In some embodiments, as used herein, the term “pharmaceutically acceptable” means approved by a regulatory agency of the Federal or a state government or listed in the U.S. Pharmacopoeia or other generally recognized pharmacopoeia for use in animals, and more particularly in humans. The phrase “pharmaceutically acceptable salt(s)”, as used herein, includes those salts of compounds of the disclosure that are safe and effective for use in mammals and that possess the desired biological activity. Pharmaceutically acceptable salts include salts of acidic or basic groups present in compounds of the disclosure or in compounds identified pursuant to the methods of the disclosure. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, and pamoate (i.e., 1,1′-methylene-bis-(2-hydroxy-3-naphthoate)) salts. Certain compounds of the disclosure can form pharmaceutically acceptable salts with various amino acids. Suitable base salts include, but are not limited to, aluminum, calcium, lithium, magnesium, potassium, sodium, zinc, iron and diethanolamine salts. Pharmaceutically acceptable base addition salts are also formed with amines, such as organic amines. Examples of suitable amines are N,N′-dibenzylethylenediamine, chloroprocaine, choline, diethanolamine, dicyclohexylamine, ethylenediamine, N-methylglucamine, and procaine.

As used herein, the term “therapeutic” means an agent utilized to treat, combat, ameliorate, protect against, or improve an unwanted condition or disease of a subject.

A “therapeutically effective amount” or “effective amount” of a compound, such as vapendavir, or composition of the disclosure is a predetermined amount which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect or physician observes a change). The effect contemplated herein includes medical therapeutic treatment, as appropriate. The specific dose of a compound administered according to this disclosure to obtain therapeutic effects will, of course, be determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the subject: the time of administration, route of administration, and rate of excretion of the specific compound employed and the duration of the treatment. The effective amount administered will be determined by the physician in the light of the foregoing relevant circumstances and the exercise of sound medical judgment. A therapeutically effective amount of a compound of this disclosure, such as vapendavir, is typically an amount such that when it is administered in a physiologically tolerable excipient composition, it is sufficient to achieve an effective systemic concentration or local concentration in the tissue.

The terms “treat”. “treated”, or “treating” as used herein refers to therapeutic treatment measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms: diminishment of the extent or vigor or rate of development of the condition, disorder or disease: stabilization (i.e., not worsening) of the state of the condition, disorder or disease: delay in onset or slowing of the progression of the condition, disorder or disease: amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

As used herein, the term “adverse event” refers to any untoward medical occurrence associated with the use of a drug in humans, whether or not considered drug related. The abbreviation “AE” may be used to refer to an adverse event.

As used herein, the abbreviation “ADL” means activities of daily living.

As used herein, the abbreviation “ALT” means alanine aminotransferase.

As used herein, the abbreviation “AUC” means area under the curve.

As used herein, the abbreviation “BID” means bis in de and refers to twice a day dosage.

As used herein, the abbreviation “BL” means baseline.

As used herein, the abbreviation “CF” means cystic fibrosis.

As used herein, the abbreviation “Cₘₐₓ” means peak drug concentration.

As used herein, the abbreviation “IPF” means Idiopathic Pulmonary Fibrosis.

As used herein, the abbreviation “CYP3A2” means Cytochrome P450 family 1 subfamily A member 2.

As used herein, the abbreviation “CYP3A4” means Cytochrome P450 family 3 subfamily A member 4.

As used herein, the abbreviation “CXCL8” means C—X—C motif chemokine ligand 8.

As used herein, the abbreviation “CXCL10” means C—X—C motif chemokine ligand 10.

As used herein, “EC₅₀” refers to half maximal effective concentration.

As used herein, “EC₉₀” refers to 90% effective concentration.

As used herein, “paEC₅₀” refers to the protein adjusted half maximal effective concentration.

As used herein, “paEC₉₀” refers to the protein adjusted 90% effective concentration.

As used herein, “FEV₁” refers to Forced Expiratory Volume in 1 second.

As used herein, “FVC” means Forced Vital Capacity.

Unless otherwise indicated, the abbreviation “h” refers to hours as a unit of time.

As used herein, the abbreviation “Scr” means screening.

As used herein, the abbreviation “IC” means informed consent.

As used herein, the abbreviation “MH” means medical history.

As used herein, the abbreviation “IE” means inclusion/exclusion criteria.

As used herein, the abbreviation “IC₅₀” refers to half maximal inhibitory concentration.

As used herein, the abbreviation “PT” means pregnancy test.

As used herein, the abbreviation “T” means training of diaries, unless otherwise indicated.

As used herein, the abbreviation “D” means diaries.

As used herein, the abbreviation “PE” means physical examination.

As used herein, the abbreviation “PEF” means peak expiratory flow.

As used herein, the abbreviation “PK” means pharmacokinetics.

As used herein, the abbreviation “LF” means lung function.

As used herein, the abbreviation “NL” means nasal lavage and nasosorption.

As used herein, the abbreviation “Bl” means blood chemistry and full blood count.

As used herein, the abbreviation “Bl-Ser” means RV-A16 serology, mediators.

As used herein, the abbreviation “Bl-PK” means pharmacodynamic testing of blood.

As used herein, the abbreviation “SAE” means serious adverse event.

As used herein, the abbreviation “Sp” means sputum.

As used herein, the abbreviation “SM” means safety assessment.

As used herein, the abbreviation “CM” means concomitant medications.

Disclosed herein are subject in need thereof using vapendavir. Vapendavir (3-Ethoxy-6-{2-[1-(6-methylpyridazin-3-yl) piperidin-4-yl]ethoxy}-benzo[d]isoxazol) is a potent and broad spectrum antiviral agent active against >97% of RV-A and RV-B serotypes of RV (no available assay for RV-C, but clinical data indicate sensitivity similar to A and B) and 89% of other EVs evaluated in cell based assay systems. Vapendavir acts by binding to the viral capsid, thereby inhibiting viral attachment to the target cell and, independently, preventing release of viral RNA into the cell. Vapendavir has been in development for the treatment of infections with RV and other respiratory EVs for approximately a decade, with over 640 healthy adults and participants with asthma receiving treatment across 7 clinical studies. Vapendavir reduced RV viral load compared with placebo in three Phase 2 clinical trials and reduced respiratory symptoms in a Phase 2 clinical trial, both in adult participants with asthma. An acceptable safety and tolerability profile has been demonstrated to date, with no appreciable safety concerns noted following single doses up to 1,056 milligram (mg) and multiple doses up to 528 mg bis in de/twice a day (BID) for up to 7 days, and 400 mg BID, up to 10 days in a RV challenge model. Vapendavir has also been tested against the RV-A16 human challenge strain and demonstrated complete protection against viral replication and de novo infection at ≥0.312 micromolar (μM) using in vitro HeLa cell assays. Vapendavir may also be referred to as VPV. The chemical structure (Formula (I)) of vapendavir is shown below.

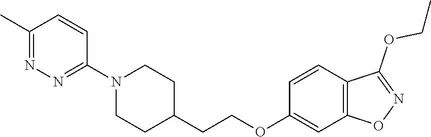 [SMILES: CCOc1noc2cc(OCCC3CCN(c4ccc(C)nn4)CC3)ccc12]

In some embodiments, there is provided a method of treating a respiratory enterovirus infection in a human subject in need thereof, which in some embodiments includes orally administering to the subject a therapeutically effective amount of vapendavir or a pharmaceutically acceptable salt thereof, thereby treating the respiratory enterovirus infection in the subject. In any of the embodiments described herein, the respiratory enterovirus infection may be a respiratory enterovirus infection. The respiratory enterovirus infection may be, for example, or combinations thereof.

In some embodiments, there is provided a method of treating a respiratory enterovirus infection in a human subject, with includes orally administering to the subject a therapeutically effective amount of vapendavir or a pharmaceutically acceptable salt thereof, thereby treating the respiratory enterovirus infection in the subject: wherein the therapeutically effective amount of vapendavir or a pharmaceutically acceptable salt thereof is between about 250 mg and about 2,000 mg per day

In some embodiments, the respiratory enterovirus infection is acute, and in other embodiments, the respiratory enterovirus infection is not acute. In some embodiments, the respiratory enterovirus is selected from a rhinovirus, echovirus, EV-68, EV-71, coxsackie virus, a non-polio enterovirus and combinations thereof.

In some embodiments, the therapeutically effective amount of vapendavir or a pharmaceutically acceptable salt thereof is between about 250 mg and about 2,000 mg per day, such as about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,650 mg, about 1,700 mg, about 1,750 mg, about 1,800 mg, about 1,850 mg, about 1,900 mg, about 1,950 mg, about 2,000 mg, or any range or value contained within these values.

In some embodiments, the therapeutically effective amount of vapendavir or a pharmaceutically acceptable salt thereof is between about 1 mg/kg and about 75 mg/kg, relative to the body weight of the subject. For example, the therapeutically effective amount of vapendavir or a pharmaceutically acceptable salt thereof is, in some embodiments, about 1 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60) mg/kg, about 65 mg/kg, about 70 mg/kg, about 75 mg/kg, or any range or value contained therein. In some embodiment, the therapeutically effective amount of vapendavir or a pharmaceutically acceptable salt thereof is about 14 mg/kg. In some embodiment, the therapeutically effective amount of vapendavir or a pharmaceutically acceptable salt thereof is about 7 mg/kg. In some embodiment, the therapeutically effective amount of vapendavir or a pharmaceutically acceptable salt thereof is about 3.5 mg/kg.

In some embodiments, the therapeutically effective amount of vapendavir or a pharmaceutically acceptable salt thereof is an amount that achieves a Cₘₐₓ of about 2000 to about 12000 ng/ml. In some embodiments, the therapeutically effective amount of vapendavir or a pharmaceutically acceptable salt thereof is an amount that achieves a Cₘₐₓ of about 2000 to about 6000 ng/ml. In some embodiments, the therapeutically effective amount of vapendavir or a pharmaceutically acceptable salt thereof is an amount that achieves a Cₘₐₓ of about 2000 to about 4000 ng/ml. In some embodiments, the therapeutically effective amount of vapendavir or a pharmaceutically acceptable salt thereof is an amount that achieves a Cₘₐₓ of about 3000 to about 6000 ng/ml. In some embodiments, the therapeutically effective amount of vapendavir or a pharmaceutically acceptable salt thereof is an amount that achieves a Cₘₐₓ of about 6000 to about 8000 ng/ml. In some embodiments, the therapeutically effective amount of vapendavir or a pharmaceutically acceptable salt thereof is an amount that achieves a Cₘₐₓ of about 8000 to about 10000 ng/ml. In some embodiments, the therapeutically effective amount of vapendavir or a pharmaceutically acceptable salt thereof is an amount that achieves a Cₘₐₓ of about 10000 to about 12000 ng/ml.

In some embodiments, the therapeutically effective amount of vapendavir or a pharmaceutically acceptable salt thereof is a single loading dose of about 1,000 mg followed by a maintenance dose of about 500 mg about every 12 hours. In some embodiments, the therapeutically effective amount of vapendavir or a pharmaceutically acceptable salt thereof is a single loading dose of about 1,000 mg followed by a maintenance dose of about 500 mg about every 12 hours for about 4 to about 10 days. In some embodiments, the therapeutically effective amount of vapendavir or a pharmaceutically acceptable salt thereof is a single loading dose of about 1,000 mg followed by a maintenance dose of about 500 mg about every 12 hours for about 5 to about 8 days. In some embodiments, the therapeutically effective amount of vapendavir or a pharmaceutically acceptable salt thereof is a single loading dose of about 1,000 mg followed by a maintenance dose of about 500 mg every 12 hours for about 6 to about 8 days. In some embodiments, the therapeutically effective amount of vapendavir or a pharmaceutically acceptable salt thereof is a single loading dose of about 1,000 mg followed by a maintenance dose of about 500 mg every 12 hours for about 7 days.

In some embodiments, the therapeutically effective amount of vapendavir or a pharmaceutically acceptable salt thereof is a single loading dose of about 1,000 mg followed by a maintenance dose of about 500 mg every 12 hours. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed by a maintenance dose of about 500 mg every 12 hours for about 5 to about 8 days. In some embodiments, the therapeutically effective amount of vapendavir or a pharmaceutically acceptable salt thereof is a single loading dose of about 1,000 mg followed by a maintenance dose of about 500 mg every 12 hours for about 6 to about 8 days. In some embodiments, the therapeutically effective amount of vapendavir or a pharmaceutically acceptable salt thereof is a single loading dose of about 1,000 mg followed by a maintenance dose of about 500 mg every 12 hours for about 7 days.

In some embodiments, the therapeutically effective amount of vapendavir or a pharmaceutically acceptable salt thereof is a single loading dose of about 1000 mg followed by a maintenance dose of about 500 mg every 12 hours for about 4 days. In some embodiments, the therapeutically effective amount of vapendavir or a pharmaceutically acceptable salt thereof is a single loading dose of about 1000 mg followed by a maintenance dose of about 500 mg every 12 hours for about 5 days. In some embodiments, the therapeutically effective amount of vapendavir or a pharmaceutically acceptable salt thereof is a single loading dose of about 1000 mg followed by a maintenance dose of about 500 mg every 12 hours for about 6 days. In some embodiments, the therapeutically effective amount of vapendavir or a pharmaceutically acceptable salt thereof is a single loading dose of about 1000 mg followed by a maintenance dose of about 500 mg every 12 hours for about 7 days. In some embodiments, the therapeutically effective amount of vapendavir or a pharmaceutically acceptable salt thereof is a single loading dose of about 1000 mg followed by a maintenance dose of about 500 mg every 12 hours for about 8 days. In some embodiments, the therapeutically effective amount of vapendavir or a pharmaceutically acceptable salt thereof is a single loading dose of about 1000 mg every 12 hours for one day followed by a maintenance dose of about 500 mg every 12 hours for about 9 days. In some embodiments, the therapeutically effective amount of vapendavir or a pharmaceutically acceptable salt thereof is a single loading dose of about 1000 mg followed by a maintenance dose of about 500 mg every 12 hours for 10 days.

In some embodiments, the therapeutically effective amount of vapendavir or a pharmaceutically acceptable salt thereof is a single loading dose of about 1000 mg followed by a maintenance dose of about 500 mg about every 12 hours for about 4 days. In some embodiments, the therapeutically effective amount of vapendavir or a pharmaceutically acceptable salt thereof is a single loading dose of about 1000 mg followed by a maintenance dose of about 500 mg about every 12 hours for about 5 days. In some embodiments, the therapeutically effective amount of vapendavir or a pharmaceutically acceptable salt thereof is a single loading dose of about 1000 mg followed by a maintenance dose of about 500 mg every 12 hours for about 6 days. In some embodiments, the therapeutically effective amount of vapendavir or a pharmaceutically acceptable salt thereof is a single loading dose of about 1000 mg followed by a maintenance dose of about 500 mg about every 12 hours for about 7 days. In some embodiments, the therapeutically effective amount of vapendavir or a pharmaceutically acceptable salt thereof is a single loading dose of about 1000 mg followed by a maintenance dose of about 500 mg about every 12 hours for about 8 days. In some embodiments, the therapeutically effective amount of vapendavir or a pharmaceutically acceptable salt thereof is a single loading dose of about 1000 mg per day followed by a maintenance dose of about 500 mg about every 12 hours for about 9 days. In some embodiments, the therapeutically effective amount of vapendavir or a pharmaceutically acceptable salt thereof is a single loading dose of about 1000 mg followed by a maintenance dose of about 500 mg about every 12 hours for 10 days.

In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is a single loading dose of about 1,000 mg followed by a maintenance dose of about 500 mg about every 12 hours for a total of about 13 maintenance doses. In some embodiments, the therapeutically effective amount of vapendavir or a pharmaceutically acceptable salt thereof is a single loading dose of about 1,000 mg followed by a maintenance dose of about 500 mg about every 12 hours for a total of 13 maintenance doses. In some embodiments, the therapeutically effective amount of vapendavir or a pharmaceutically acceptable salt thereof is a single loading dose of about 1,000 mg followed a maintenance dose of about 500 mg every 12 hours for a total of 13 maintenance doses.

In some embodiments, the therapeutically effective amount of vapendavir or a pharmaceutically acceptable salt thereof is a single loading dose of about 14 mg/kg for one day followed by a maintenance dose of about 7 mg/kg every 12 hours for about 4 to about 10 days. In some embodiments, the therapeutically effective amount of vapendavir or a pharmaceutically acceptable salt thereof is a single loading dose of about 14 mg/kg for one day followed by a maintenance dose of about 7 mg/kg every 12 hours for about 4 days. In some embodiments, the therapeutically effective amount of vapendavir or a pharmaceutically acceptable salt thereof is a single loading dose of about 14 mg/kg for one day followed by a maintenance dose of about 7 mg/kg every 12 hours for about 5 days. In some embodiments, the therapeutically effective amount of vapendavir or a pharmaceutically acceptable salt thereof is a single loading dose of about 14 mg/kg for one day followed by a maintenance dose of about 7 mg/kg every 12 hours for about 6 days. In some embodiments, the therapeutically effective amount of vapendavir or a pharmaceutically acceptable salt thereof is a single loading dose of about 14 mg/kg for one day followed by a maintenance dose of about 7 mg/kg every 12 hours for about 7 days. In some embodiments, the therapeutically effective amount of vapendavir or a pharmaceutically acceptable salt thereof is a single loading dose of about 14 mg/kg for one day followed by a maintenance dose of about 7 mg/kg every 12 hours for about 8 days. In some embodiments, the therapeutically effective amount of vapendavir or a pharmaceutically acceptable salt thereof is a single loading dose of about 14 mg/kg for one day followed by a maintenance dose of about 7 mg/kg every 12 hours for about 9 days. In some embodiments, the therapeutically effective amount of vapendavir or a pharmaceutically acceptable salt thereof is a single loading dose of about 14 mg/kg for one day followed by a maintenance dose of about 7 mg/kg every 12 hours for about 10 days. In some embodiments, the therapeutically effective amount of vapendavir or a pharmaceutically acceptable salt thereof is a single loading dose of about 14 mg/kg for one day followed by a maintenance dose of about 7 mg/kg every 12 hours for a total of about 13 maintenance doses.

In some embodiments, the therapeutically effective amount of vapendavir or a pharmaceutically acceptable salt thereof is a single loading dose of about 14 mg/kg for about one day followed by a maintenance dose of about 7 mg/kg about every 12 hours for about 4 to about 10 days. In some embodiments, the therapeutically effective amount of vapendavir or a pharmaceutically acceptable salt thereof is a single loading dose of about 14 mg/kg for about one day followed by a maintenance dose of about 7 mg/kg about every 12 hours for about 4 days. In some embodiments, the therapeutically effective amount of vapendavir or a pharmaceutically acceptable salt thereof is a single loading dose of about 14 mg/kg for about one day followed by a maintenance dose of about 7 mg/kg about every 12 hours for about 5 days. In some embodiments, the therapeutically effective amount of vapendavir or a pharmaceutically acceptable salt thereof is a single loading dose of about 14 mg/kg for about one day followed by a maintenance dose of about 7 mg/kg about every 12 hours for about 6 days. In some embodiments, the therapeutically effective amount of vapendavir or a pharmaceutically acceptable salt thereof is a single loading dose of about 14 mg/kg for about one day followed by a maintenance dose of about 7 mg/kg every 12 hours for about 7 days. In some embodiments, the therapeutically effective amount of vapendavir or a pharmaceutically acceptable salt thereof is a single loading dose of about 14 mg/kg for about one day followed by a maintenance dose of about 7 mg/kg about every 12 hours for about 8 days. In some embodiments, the therapeutically effective amount of vapendavir or a pharmaceutically acceptable salt thereof is a single loading dose of about 14 mg/kg for about one day followed by a maintenance dose of about 7 mg/kg about every 12 hours for about 9 days. In some embodiments, the therapeutically effective amount of vapendavir or a pharmaceutically acceptable salt thereof is a single loading dose of about 14 mg/kg for about one day followed by a maintenance dose of about 7 mg/kg about every 12 hours for about 9 days. In some embodiments, the therapeutically effective amount of vapendavir or a pharmaceutically acceptable salt thereof is a single loading dose of about 14 mg/kg for about one day followed by a maintenance dose of about 7 mg/kg about every 12 hours for a total of about 13 maintenance doses.

In some embodiments, the dosage regimen of vapendavir or a pharmaceutically acceptable salt thereof could be varied. For instance, the single loading dose could be adjusted within a range of about 500 mg to about 1500 mg, followed by a maintenance dose of about 250 mg to about 750 mg every 12 hours for about 3 to about 14 days. The specific dosage could be adjusted based on the patient's weight, age, severity of the COPD, and the specific strain of the respiratory enterovirus infection.

In some embodiments, the single loading dose is administered within about 24 hours of the subject displaying symptoms of a respiratory enterovirus infection. In some embodiments, the single loading dose is administered within about 48 hours of the subject displaying symptoms of a respiratory enterovirus infection. In some embodiments, the single loading dose is administered within about 72 hours of the subject displaying symptoms of a respiratory enterovirus infection. In some embodiments, the single loading dose is administered within about 96 hours of the subject displaying symptoms of a respiratory enterovirus infection. In some embodiments, the single loading dose is administered within about 72 hours to about 96 hours of the subject displaying symptoms of a respiratory enterovirus infection. In some embodiments, the single loading dose is administered within about 1 to about 5 days of the subject displaying symptoms of a respiratory enterovirus infection. In some embodiments, the single loading dose is administered within about 1 to about 7 days of the subject displaying symptoms of a respiratory enterovirus infection. In some embodiments, the single loading dose is administered greater than 48 hours after the subject first displays symptoms of a respiratory enterovirus infection. In some embodiments, the single loading dose is administered prior to the subject achieving a peak viral load.

In some embodiments, administration of single loading dose of about 1000 mg results in a Cmax of about 2150 ng/ml.

In some embodiments, the symptoms of the respiratory enterovirus infection may include, a cough, increased mucus production, sneezing, nasal congestion, runny nose, sore throat, headaches, muscle aches, breathlessness, tight chest, wheezing, fever, red eyes, swelling of the lymph nodes, fatigue, hoarse voice, rhinorrhea, malaise, nasal obstruction, sputum production, inability to function at normal level of activity, and any combinations thereof. In some embodiments, the subject is asymptomatic but has been diagnosed with a respiratory enterovirus infection.

In some embodiments, the maintenance dose is first administered about 6 to about 12 hours following administering the single loading dose. In some embodiments, the maintenance dose may be administered about 6 hours, about 7 hours, about 8 hours, about 9 hours, about 10 hours, about 11 hours, about 12 hours, or any range or value contained therein, after administration of the single loading dose. In some embodiments, the at least one maintenance dose is administered for about 6 days to about 10 days, such as about 6 days, about 7 days, about 8 days, about 9 days, or about 10 days.

In some embodiments, the single loading dose and the maintenance dose are administered within about 30 minutes of the subject consuming a meal with solid food (e.g. the subject is in a fed state). In some embodiments, the single loading dose and the maintenance dose are administered within about 30 minutes of the subject consuming a high fat meal with solid food (e.g. the subject is in a fed state). In some embodiments, administering the single loading dose and maintenance dose to a subject within about 30 minutes of the subject consuming a meal with solid food results in a higher plasma concentration of vapendavir or a pharmaceutically acceptable salt thereof than administering the single loading dose and maintenance dose to a subject in a fasted state. For example, administration of 500 mg vapendavir in normal healthy volunteers that were in a fed state prior to administration results in a Cₘₐₓ of 2060 ng/ml (CV % 5 ng/ml) compared to 687 ng/ml (CV % 93 ng/ml) in normal healthy volunteers that were in fasted state prior to administration.

In some embodiments, the single loading dose is administered after the subject has been diagnosed with a respiratory enterovirus infection, wherein the diagnosis is achieved by a clinical diagnosis, multiplex PCR testing, rapid antigen testing, direct fluorescent antibody testing, arterial blood gas testing, chest x-rays, peak flow meter testing, pleural fluid cultures, lung CT scanning, lung function tests, pulse oximetry testing, spirometry testing, sputum testing, nasal swab testing, throat swab testing, or any combination thereof.

In some embodiments, the maintenance dose of vapendavir or a pharmaceutically acceptable salt thereof is a dose of about 500 mg administered about every 12 hours for about 6 to about 10 days and is administered to the subject in a fed state. In some embodiments, the maintenance dose of vapendavir or a pharmaceutically acceptable salt thereof is a dose of about 500 mg administered about every 12 hours for about 4 days and is administered to the subject in a fed state. In some embodiments, the maintenance dose of vapendavir or a pharmaceutically acceptable salt thereof is a dose of about 500 mg administered about every 12 hours for about 5 days and is administered to the subject in a fed state. In some embodiments, the maintenance dose of vapendavir or a pharmaceutically acceptable salt thereof is a dose of about 500 mg administered about every 12 hours for about 6 days and is administered to the subject in a fed state. In some embodiments, the maintenance dose of vapendavir or a pharmaceutically acceptable salt thereof is a dose of about 500 mg administered about every 12 hours for about 7 days and is administered to the subject in a fed state. In some embodiments, the maintenance dose of vapendavir or a pharmaceutically acceptable salt thereof is a dose of about 500 mg administered about every 12 hours for about 8 days and is administered to the subject in a fed state. In some embodiments, the maintenance dose of vapendavir or a pharmaceutically acceptable salt thereof is a dose of about 500 mg administered about every 12 hours for about 9 days and is administered to the subject in a fed state. In some embodiments, the maintenance dose of vapendavir or a pharmaceutically acceptable salt thereof is a dose of about 500 mg administered about every 12 hours for about 10 days and is administered to the subject in a fed state.

In some embodiments, a first dose of about 1000 mg per day is administered to the subject within about 24 hours, about 48 hours, about 72 hours, or about 1 day to about 5 days of the subject displaying symptoms of a respiratory enterovirus infection. In some embodiments, the first dose is administered after the subject has been diagnosed with a respiratory enterovirus infection, wherein the diagnosis is achieved by a clinical diagnosis, multiplex PCR testing, rapid antigen testing, direct fluorescent antibody testing, arterial blood gas testing, chest x-rays, peak flow meter testing, pleural fluid cultures, lung CT scanning, lung function tests, pulse oximetry testing, spirometry testing, sputum testing, nasal swab testing, throat swab testing. In some embodiments, the symptoms of a respiratory enterovirus infection are selected from a cough, increased mucus production, sneezing, nasal congestion, runny nose, sore throat, headaches, muscle aches, breathlessness, tight chest, wheezing, fever, red eyes, swelling of the lymph nodes, fatigue, hoarse voice, rhinorrhea, malaise, nasal obstruction, sputum production, inability to function at normal level of activity, and any combinations thereof. In some embodiments, the subject is asymptomatic but has been diagnosed with a respiratory enterovirus infection.

In some embodiments, the subject is a human. In some embodiments, the subject is in a fed state. In some embodiments the subject is a human under the age of about 18 years. In some embodiments the subject is a human under the age of about 12 years. In some embodiments the subject is a human under the age of about 6 years. In some embodiments, the subject is a human between about 40 and about 85 years of age. The subject may have an underlying condition, in some embodiments of the present disclosure. In some embodiments, the underlying condition is selected from chronic obstructive pulmonary diseases (COPD), asthma, mixed COPD and asthma, emphysema: cystic fibrosis (CF): bronchiolitis obliterans; fibrosis such as tuberculosis (TB), idiopathic pulmonary fibrosis (IPF), other major respiratory diagnosis (e.g., pneumonia, aspergillosis), non-CF bronchiectasis, COPD due to alpha-1 antitrypsin deficiency, active allergic rhinitis, nasal disease, (e.g. nasal polyposis, significant septal deviation, chronic rhinosinusitis, etc.); primary ciliary dyskinesia: bronchitis: pulmonary hypertension: pulmonary edema: acute respiratory distress syndrome (ARDS); pneumoconiosis: interstitial lung disease: mesothelioma: sickle cell disease, cancer, or any combination thereof. In some embodiments, the subject has chronic obstructive pulmonary disease (COPD) and the subject been diagnosed with Global Initiative for Chronic Obstructive Lung Disease (GOLD) stage 1 COPD, GOLD stage 2 COPD, GOLD stage 3 COPD, or GOLD stage 4 COPD.

In some embodiments, the subject is a female receiving an oral contraceptive, such as birth control. In such embodiments, administering a therapeutically effective amount of vapendavir or a pharmaceutically acceptable salt thereof further includes administering additional birth control during treatment. In some embodiments, the subject is a female that is not receiving an oral contraceptive, such as birth control.

In some embodiments, the subject is receiving stable maintenance therapy for COPD. In some embodiments, stable maintenance therapy for COPD may include short-acting bronchodilators such as, but not limited to Albuterol, Levalbuterol, Ipratropium, or any combination thereof: long-acting bronchodilators such as, but not limited to Aclidinium, Arformoterol, Formoterol, Glycopyrrolate, Indacaterol, Olodaterol, Revefenacin, Salmeterol, Tiotropium, Umeclidinium, or any combination thereof: corticosteroids such as, but not limited to Fluticasone. Budesonide, Prednisolone, Beclometasone or any combination thereof: LABA and LAMA combination bronchodilator therapies such as, but not limited to Aclidinium/formoterol, Glycopyrrolate/formoterol, Tiotropium/olodaterol, Umeclidinium/vilanterol, Glycopyrronium/indacaterol or any combination thereof; Combinations of an ICS and a long-acting bronchodilator such as, but not limited to Budesonide/formoterol, Fluticasone/salmeterol. Fluticasone/vilanterol, Beclometasone/formoterol or any combination thereof: Triple Combination Therapies (ICS/LAMA/LABA) such as but not limited to Fluticasone/umeclidinium/vilanterol, Budesonide/glycopironium/formoterol, Beclometasone/formoterol/glycopyrronium or any combination thereof: Methylxanthines such as, but not limited to Theophylline; Phosphodiesterase-4 inhibitors such as, but not limited to Roflumilast: Mucoactive drugs such as but not limited to Carbocysteine, Erdosteine, N-acetylcysteine or any combination thereof; Biologics/anti-eosinophilia drugs such as, but not limited to Mepolizumab, Benralizumab, Reslizumab, Dupilumab or any combination thereof: or any other drug prescribed for the treatment of COPD, may receive the same stable maintenance therapy for COPD than when the subject does not have a respiratory enterovirus infection. In some embodiments, the subject may receive a larger dose, a more frequent dosage, or any combination thereof, of the stable maintenance therapy for COPD than when the subject does not have a respiratory enterovirus infection. In some embodiments, the subject may receive a smaller dose, a less frequent dosage, or any combination thereof, of the stable maintenance therapy for COPD than when the subject does not have a respiratory enterovirus infection.

The subject may, in some embodiments, have had at least one respiratory enterovirus infection in the 12 months immediately prior to administering the therapeutically effective amount of vapendavir or a pharmaceutically acceptable salt thereof. In some embodiments, the subject has not had a sore throat, sneezing, rhinorrhea, malaise, nasal obstruction, cough, or any combination thereof, in the 30 days prior to administering the therapeutically effective amount of vapendavir or a pharmaceutically acceptable salt thereof. In some embodiments, the subject does not have an active diagnosed infection or consistent symptoms thereof, with a viral or bacterial pathogen in addition to respiratory enterovirus infection.

In some embodiments, the subject has not been diagnosed with asthma: cystic fibrosis (CF): bronchiolitis obliterans: fibrosis such as tuberculosis (TB), idiopathic pulmonary fibrosis (IPF), other major respiratory diagnosis (e.g., pneumonia, aspergillosis), non-CF bronchiectasis, COPD due to alpha-1 antitrypsin deficiency, active allergic rhinitis, nasal disease. (e.g. nasal polyposis, significant septal deviation, chronic rhinosinusitis, etc.) or any combination thereof.

In some embodiments, the subject is not taking any medications that are known to be impacted by or significantly metabolized by CYP3A4. Such medications include but are not limited to Apalutamide, Amiodarone, Carbamazepine, Amprenavir, Dexamethasone, Atazanavir, Enzalutamide, Boceprevir, Fosphenytoin, Clarithromycin, Lumacaftor, Cobicistat, Midostaurin, Conivaptan, Mitotane, Curcumin, Pentobarbital, Danazol, Phenobarbital, Danoprevir, Phenytoin, Darunavir, Primidone, Delavirdine, Rifampicin, Diltiazem, Rifamycin, Ditiocarb, Rifapentine, Econazole, Rifaximin, Efavirenz, Rimexolone, Elvitegravir, St. John's Wort, Ergotamine, Idelalisib, Levoketoconazole, Naloxone, Indinavir, Itraconazole, Ketoconazole, Lonafarnib, Loperamide, Lopinavir, Methimazole, Midostaurin, Nefazodone, Nelfinavir, Nilotinib, Posaconazole, Ribociclib, Ritonavir, Saquinavir, Stiripentol, Telaprevir, Telithromycin, Terfenadine, Tipranavir, Troleandomycin, Voriconazole, or combinations thereof. Such medications may also include H2 blockers such as Famotidine, Cimetidine, Nizatidine and Ranitidine. Such medications may also include proton pump inhibitors such as but not limited to Omeprazole, Esomeprazole, Lansoprazole, Rabeprazole, Pantoprazole, Dexlansoprazole and Zegerid. Such medications may also antacids such a Melox (Aluminum hydroxide, Magnesium hydroxide and Simethicone), Alginate, Aluminum hydroxide, Calcium carbonate, Magnesium carbonate, Magnesium hydroxide, Magnesium trisilicate, and Sodium bicarbonate.

In some embodiments, treating the respiratory enterovirus infection in the subject includes decreasing and/or preventing an increase in one or more parameters or measurements known to those skilled in the art to be indicative of a subject's health.

In some embodiments, treating the respiratory enterovirus infection in the subject includes decreasing a viral load in the sputum, nasal passages, or a combination thereof in the subject compared with a viral load in the sputum, nasal passages, or a combination thereof of the subject prior to treatment.

Treating the respiratory enterovirus infection in the subject may include, in some embodiments, decreasing the subjects' Evaluating Respiratory Symptoms in COPD Score (E-RS), including the subjects' Evaluating Respiratory Symptoms in COPD Score (E-RS), compared to the subjects' Evaluating Respiratory Symptoms in COPD Score (E-RS) prior to treatment. In some embodiments, treating the respiratory enterovirus infection in the subject includes preventing an increase in the subjects Evaluating Respiratory Symptoms in COPD Score (E-RS) compared to the subjects' Evaluating Respiratory Symptoms in COPD Score (E-RS) score prior to treatment.

In some embodiments, treating the respiratory enterovirus infection in the subject includes decreasing the subjects' peak lower respiratory symptom score (LRSS), such as peak lower respiratory symptom score (LRSS), compared to the subjects' peak lower respiratory symptom score (LRSS) prior to treatment. Similarly, treating the respiratory enterovirus infection in the subject may include preventing an increase in the subjects' LRSS compared to the subjects' LRSS prior to treatment. In some embodiments, treating the respiratory enterovirus infection in the subject includes decreasing and/or preventing an increase in the subjects peak upper respiratory symptom score (URSS), such as URSS, compared to the subjects' peak upper respiratory symptom score (URSS) prior to treatment.

In some embodiments, treating the respiratory enterovirus infection in the subject includes decreasing the subjects EXAcerbation of Chronic Pulmonary Disease Tool-Patient Reported Outcome (EXACT-PRO), such as the subjects' EXAcerbation of Chronic Pulmonary Disease Tool-Patient Reported Outcome (EXACT-PRO) score, compared to the subjects' EXAcerbation of Chronic Pulmonary Disease Tool-Patient Reported Outcome (EXACT-PRO) prior to treatment. Similarly, treating the respiratory enterovirus infection in the subject may include preventing an increase in the subjects EXACT-PRO, such as the subjects EXACT-PRO score. In some embodiments, treating the respiratory enterovirus infection in the subject includes preventing an increase in the subjects EXAcerbation of Chronic Pulmonary Disease Tool-Patient Reported Outcome (EXACT-PRO), such as the subjects' EXAcerbation of Chronic Pulmonary Disease Tool-Patient Reported Outcome (EXACT-PRO) score, compared to the subjects EXAcerbation of Chronic Pulmonary Disease Tool-Patient Reported Outcome (EXACT-PRO) prior to treatment. Similarly, treating the respiratory enterovirus infection in the subject may include preventing an increase in the subjects EXACT-PRO, such as the subjects EXACT-PRO score.

In some embodiments, treating the respiratory enterovirus infection in the subject includes decreasing the subjects EXAcerbation of Chronic Pulmonary Disease Tool-Patient Respiratory Symptoms (EXACT-RS), such as EXAcerbation of Chronic Pulmonary Disease Tool-Patient Respiratory Symptoms (EXACT-RS) score, compared to the subjects' EXAcerbation of Chronic Pulmonary Disease Tool-Patient Respiratory Symptoms (EXACT-RS) prior to treatment. In some embodiments, treating the respiratory enterovirus infection in the subject includes preventing an increase in the subjects' EXACT-RS score. In some embodiments, treating the respiratory enterovirus infection in the subject includes preventing an increase in the subjects EXAcerbation of Chronic Pulmonary Disease Tool-Patient Respiratory Symptoms (EXACT-RS), such as EXAcerbation of Chronic Pulmonary Disease Tool-Patient Respiratory Symptoms (EXACT-RS) score, compared to the subjects' EXAcerbation of Chronic Pulmonary Disease Tool-Patient Respiratory Symptoms (EXACT-RS) prior to treatment. In some embodiments, treating the respiratory enterovirus infection in the subject includes preventing an increase in the subjects' EXACT-RS score.

In some embodiments, treating the respiratory enterovirus infection in the subject includes decreasing and/or preventing an increase in the subjects' COPD assessment tool (CAT) score compared to the subjects' COPD assessment tool (CAT) score prior to treatment.

In some embodiments, treating the respiratory enterovirus infection in the subject includes decreasing the subjects' St. George's Respiratory Questionnaire (SGRQ) score compared to the subjects' St. George's Respiratory Questionnaire (SGRQ) score prior to treatment. In some embodiments, treating the respiratory enterovirus infection in the subject includes preventing an increase in the subjects' St. George's Respiratory Questionnaire (SGRQ) score compared to the subjects' St. George's Respiratory Questionnaire (SGRQ) score prior to treatment.

In some embodiments, treating the respiratory enterovirus infection in the subject includes decreasing and/or preventing an increase in the subjects' Wisconsin Upper Respiratory Symptom Survey (WURSS) score compared to the subjects' Wisconsin Upper Respiratory Symptom Survey (WURSS) score prior to treatment.

In some embodiments, treating the respiratory enterovirus infection in the subject includes improving the subject's lung function compared to the subjects' lung function prior to treatment. The subjects' lung function may, in some embodiments, be measured by methods familiar to those skilled in the art, including Forced Expiratory Volume-1 (FEV₋₁: in liters and % predicted), Forced Vital Capacity (FVC: in liters and % predicted) FEV₁/FVC ratio and peak expiratory flow (PEF), or any combination thereof.

In some embodiments, treating the respiratory enterovirus infection in the subject includes preventing a decrease in the subjects' lung function compared to the subjects' lung function prior to treatment, wherein the subjects' lung function is measured by Forced Expiratory Volume-1 (FEV₋₁: in liters and % predicted), Forced Vital Capacity (FVC: in liters and % predicted) FEV 1/FVC ratio and peak expiratory flow (PEF), or any combination thereof.

In some embodiments, treating the respiratory enterovirus infection in the subject includes reducing the subjects' peak nasal lavage viral load compared to the subjects' peak nasal lavage viral load prior to treatment. In some embodiments, treating the respiratory enterovirus infection in the subject includes preventing an increase in the subjects' peak nasal lavage viral load compared to the subjects' peak nasal lavage viral load prior to treatment.

In some embodiments, treating the respiratory enterovirus infection in the subject includes reducing and/or preventing an increase in the subjects' peak sputum lavage viral load compared to the subjects' peak sputum lavage viral load prior to treatment. In some embodiments, treating the respiratory enterovirus infection in the subject includes reducing and/or preventing bacterial bronchitis, pneumonia, or combinations thereof.

In some embodiments, treating the respiratory enterovirus infection in the subject includes reducing the subjects' AUC nasal viral load compared to the subjects' AUC nasal viral load prior to treatment, and in some embodiments, treating the respiratory enterovirus infection in the subject includes preventing an increase in the subjects' AUC nasal viral load compared to the subjects' AUC nasal viral load prior to treatment. Treating the respiratory enterovirus infection in the subject may, in some embodiments, include reducing and/or preventing an increase in the subjects' AUC sputum viral load compared to the subjects' AUC sputum viral load prior to treatment.

In some embodiments, treating the respiratory enterovirus infection in the subject includes reducing the duration of viral shedding, reducing the number of days wherein the subject is positive for bacteria in the sputum, or combinations thereof.

In some embodiments, treating the respiratory enterovirus infection in the subject includes reducing and/or preventing an increase in the subjects' peak sputum bacterial load compared to the subjects' peak sputum lavage bacterial load prior to treatment. In some embodiments, treating the respiratory enterovirus infection in the subject includes reducing and/or preventing bacterial bronchitis, pneumonia, or combinations thereof. In some embodiments, treating the respiratory enterovirus infection in the subject includes reducing and/or preventing an increase in the subjects' AUC sputum bacterial load compared to the subjects' AUC sputum bacterial load prior to treatment.

In some embodiments, treating the respiratory enterovirus infection in the subject includes preventing, or reducing, acute exacerbations of the underlying condition in the subject caused by the respiratory enterovirus infection. In some embodiments, treating the respiratory enterovirus infection in the subject includes a reduction in the frequency of acute exacerbations of the underlying condition in the subject, a reduction in the severity of acute exacerbations of the underlying condition in the subject, a reduction in the duration of acute exacerbations of the underlying condition in the subject or combinations thereof, caused by the respiratory enterovirus infection. In some embodiments, treating the respiratory enterovirus infection in the subject includes preventing an increase in the frequency of acute exacerbations of the underlying condition in the subject, an increase in the severity of acute exacerbations of the underlying condition in the subject, an increase in the duration of acute exacerbations of the underlying condition in the subject or combinations thereof, caused by the respiratory enterovirus infection.

In some embodiments, administering to the subject a therapeutically effective amount of vapendavir or a pharmaceutically acceptable salt thereof exceeds the paEC₅₀ for human rhinoviruses. In some embodiments, the paEC₅₀ for human rhinoviruses is about 75.4 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₅₀ for human rhinoviruses is exceeded for about 4 to about 10 days or until treatment is stopped.

In some embodiments, administering to the subject a therapeutically effective amount of vapendavir or a pharmaceutically acceptable salt thereof exceeds the paEC₉₀ for human rhinoviruses. In some embodiments, the paEC₉₀ for human rhinoviruses is about 678.6 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₉₀ for human rhinoviruses is exceeded for about 4 to about 10 days or until treatment is stopped.

In some embodiments, administering to the subject a therapeutically effective amount of vapendavir or a pharmaceutically acceptable salt thereof results in a Cₘₐₓ that exceeds the paEC₅₀ for human rhinoviruses. In some embodiments, the paEC₅₀ for human rhinoviruses is about 75.4 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₅₀ for human rhinoviruses is exceeded for about 4 to about 10 days or until treatment is stopped.

In some embodiments, administering to the subject a therapeutically effective amount of vapendavir or a pharmaceutically acceptable salt thereof results in a Cₘₐₓ that exceeds the paEC₉₀ for human rhinoviruses. In some embodiments, the paEC₉₀ for human rhinoviruses is about 678.6 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₉₀ for human rhinoviruses is exceeded for about 4 to about 10 days or until treatment is stopped.

In some embodiments, administering to the subject a therapeutically effective amount of vapendavir or a pharmaceutically acceptable salt thereof exceeds the paEC₅₀ for human rhinoviruses following a single dose. In some embodiments, the paEC₅₀ for human rhinoviruses is about 75.4 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₅₀ for human rhinoviruses is exceeded for about 4 to about 10 days or until treatment is stopped.

In some embodiments, administering to the subject a therapeutically effective amount of vapendavir or a pharmaceutically acceptable salt thereof exceeds the paEC₉₀ for human rhinoviruses following a single dose. In some embodiments, the paEC₉₀ for human rhinoviruses is about 678.6 ng/ml. In some embodiments, the therapeutically effective amount of vapendavir or a pharmaceutically acceptable salt thereof is a single loading dose of about 1,000 mg followed by a maintenance dose of about 500 mg every 12 hours for about 4 to about 10 days. In some embodiments, the therapeutically effective amount of vapendavir or a pharmaceutically acceptable salt thereof is a single loading dose of about 1,000 mg followed a maintenance dose of about 500 mg every 12 hours for a total of about 13 maintenance doses. In some embodiments, the therapeutically effective amount of vapendavir or a pharmaceutically acceptable salt thereof is an amount that achieves a Cₘₐₓ of about 2000 to about 12000 ng/ml. In some embodiments, the paEC₉₀ for human rhinoviruses is exceeded for about 4 to about 10 days or until treatment is stopped.

In some embodiments, the therapeutically effective amount of vapendavir or a pharmaceutically acceptable salt thereof is in tablet form. The vapendavir is, in some embodiments, a free base, maleate salt, oxalate salt, phosphate salt, sulfate salt, tartrate salt, or chloride salt. In some embodiments, the vapendavir is an amorphous form of vapendavir or a pharmaceutically acceptable salt thereof. In some embodiments, the vapendavir is a crystalline form of vapendavir or a pharmaceutically acceptable salt thereof.

In some embodiments, the therapeutically effective amount of vapendavir or a pharmaceutically acceptable salt thereof is administered in a sustained-release formulation.

In some embodiments, the therapeutically effective amount of vapendavir or a pharmaceutically acceptable salt thereof is administered in conjunction with at least one additional therapeutic agent selected from the group consisting of antipyretics, analgesics, antitussives, and decongestants.

In some embodiments, the therapeutically effective amount of vapendavir or a pharmaceutically acceptable salt thereof is administered to the subject in combination with a bronchodilator.

In some embodiments, the therapeutically effective amount of vapendavir or a pharmaceutically acceptable salt thereof is administered to the subject in combination with an inhaled corticosteroid.

In some embodiments, the therapeutically effective amount of vapendavir or a pharmaceutically acceptable salt thereof is administered to the subject in combination with a mucolytic agent.

In some embodiments, the therapeutically effective amount of vapendavir or a pharmaceutically acceptable salt thereof is administered to the subject in combination with an antibiotic therapy.

In some embodiments, the therapeutically effective amount of vapendavir or a pharmaceutically acceptable salt thereof is administered to the subject in combination with an antiviral therapy other than vapendavir.

In some embodiments, the therapeutically effective amount of vapendavir or a pharmaceutically acceptable salt thereof is administered to the subject in combination with oxygen therapy.

In some embodiments, the therapeutically effective amount of vapendavir or a pharmaceutically acceptable salt thereof is administered to the subject via a metered-dose inhaler.

In some embodiments, the therapeutically effective amount of vapendavir or a pharmaceutically acceptable salt thereof is administered to the subject via a nebulizer.

In some embodiments, the method of treating respiratory enterovirus infection with vapendavir could be applied to other chronic respiratory diseases such as asthma, cystic fibrosis, or bronchiectasis. These conditions also involve inflammation and exacerbation of symptoms due to viral infections and could potentially benefit from the antiviral properties of vapendavir.

In some embodiments, immunocompromised patients, such as those undergoing chemotherapy or those with HIV/AIDS, are at a higher risk of severe respiratory infections. The use of vapendavir could potentially reduce the severity and duration of these infections, improving the quality of life and survival rates for these patients.

In high-risk populations, such as the elderly or those with pre-existing respiratory conditions, vapendavir could potentially be used as a preventive treatment during periods of high risk for respiratory enterovirus infections, such as the cold and flu season. This could help to reduce the incidence of severe exacerbations and hospitalizations in these populations.

In some embodiments, respiratory enterovirus infections are common in children and can lead to severe complications such as bronchiolitis and pneumonia. The use of vapendavir in pediatric patients could potentially reduce the severity and duration of these infections, reducing the risk of complications and hospitalizations.

In some embodiments, respiratory infections are also common in animals, particularly in those kept in close quarters such as kennels, shelters, and farms. The use of vapendavir in veterinary medicine could potentially reduce the incidence and severity of these infections, improving animal health and reducing economic losses due to illness.

There is provided, in some aspects of the present disclosure, a pharmaceutical composition which includes a therapeutically effective amount of vapendavir or a pharmaceutically acceptable salt thereof. The vapendavir may be a free base, maleate salt, oxalate salt, phosphate salt, sulfate salt, or chloride salt. In some embodiments of the pharmaceutical composition, the vapendavir is an amorphous form of vapendavir or a pharmaceutically acceptable salt thereof. In some embodiments, the vapendavir is a crystalline form of vapendavir or a pharmaceutically acceptable salt thereof.

The routes for administration (delivery) include, but are not limited to, one or more of: oral (e.g., as a tablet, capsule, or as an ingestible solution), topical, mucosal (e.g., as a nasal spray or aerosol for inhalation), parenteral (e.g., by an injectable form), gastrointestinal, intraspinal, intraperitoneal, intramuscular, intravenous, intracerebroventricular, or other depot administration. Therefore, the compositions of the disclosure include those in a form especially formulated for the mode of administration. In certain embodiments, the pharmaceutical compositions of the disclosure are formulated in a form that is suitable for oral delivery.

The compounds of the disclosure may be formulated for administration in any convenient way for use in human or veterinary medicine and the disclosure therefore includes within its scope pharmaceutical compositions comprising a compound of the disclosure adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may include, in addition to the carrier, any suitable binder(s), lubricant(s), suspending agent(s), coating agent(s), and/or solubilizing agent(s).

There may be different composition/formulation requirements depending on the different delivery systems. It is to be understood that not all of the compounds need to be administered by the same route. Likewise, if the composition includes more than one active component, then those components may be administered by different routes. By way of example, the pharmaceutical composition of the disclosure may be formulated to be delivered using a mini pump or by a mucosal route, for example, as a nasal spray or aerosol for inhalation or ingestible solution, or parenterally in which the composition is formulated by an injectable form, for delivery by, for example, an intravenous, intramuscular or subcutaneous route. Alternatively, the formulation may be designed to be delivered by multiple routes.

Where appropriate, the pharmaceutical compositions can be administered by inhalation, by use of a skin patch, orally in the form of tablets containing excipients such as starch or lactose, or in capsules or ovules either alone or in admixture with excipients, or in the form of elixirs, solutions, or suspensions containing flavoring or coloring agents, or they can be injected parenterally, for example intravenously, intramuscularly, or subcutaneously. For buccal or sublingual administration, the compositions may be administered in the form of tablets or lozenges, which can be formulated in a conventional manner.

The pharmaceutical compositions of the present disclosure can be administered in the form of tablets, capsules, troches, ovules, elixirs, solutions, or suspensions, for immediate-, delayed-, modified-, sustained-, pulsed-, or controlled-release applications. The pharmaceutical compositions of the present disclosure may also be presented in the form of solutions, gels, syrups, or suspensions, or a dry powder for reconstitution with water or other suitable vehicle before use. Solid compositions such as tablets, capsules, lozenges, troches, pastilles, pills, boluses, powder, pastes, granules, bullets, or premix preparations may also be used. Solid and liquid compositions for oral use may be prepared according to methods well-known in the art. Such compositions may also contain one or more pharmaceutically acceptable carriers and excipients which may be in solid or liquid form.

The pharmaceutical composition may, in some embodiments, include about 250) mg of vapendavir or a pharmaceutically acceptable salt thereof. For example, in some embodiments, the pharmaceutical composition includes about 200 mg, about 210 mg, about 220 mg, about 230 mg, about 240 mg, about 250 mg, about 260 mg, about 270 mg, about 280) mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430) mg, about 440 mg, about 450 mg, about 460 mg, about 470) mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740) mg, about 750 mg, about 760 mg, about 770) mg, about 780 mg, about 790 mg, about 800 mg, about 810 mg, about 820 mg, about 830 mg, about 840 mg, about 850) mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1,000 mg of vapendavir or a pharmaceutically acceptable salt thereof, or any range or value contained therein. In some embodiments, the pharmaceutical composition is a tablet. The pharmaceutical composition may further include a diluent, a disintegrant, a surfactant, a lubricant, a color coating, or any combination thereof.

Oral preparations may optionally include various standard pharmaceutical carriers and excipients, such as binders, fillers, buffers, lubricants, glidants, dyes, disintegrants, odorants, sweeteners, surfactants, mold release agents, antiadhesive agents, and coatings. Some excipients may have multiple roles in the compositions, e.g., act as both binders and disintegrants.

In some embodiments, the diluent is selected from Cellulose Microcrystalline Silicified (PROSOLVHD90) (USP/NF), anhydrous calcium phosphate, anhydrous lactose, calcium carbonate, calcium lactate, calcium sulfate dihydrate, corn starch, fructose, kaolin, lactose monohydrate, magnesium hydroxide, maltitol, maltose monohydrate, mannitol, sorbitol, sucrose, tribasic calcium phosphate, sodium citrate, glycine, croscarmellose sodium, ethanol, propylene glycol, glycerin, polyvinylpyrrolidone, hydroxypropylmethylcellulose (HPMC), hydroxypropylcellulose (HPC), sucrose, gelatin, acacia, or any combination thereof.

In some embodiments, the disintegrant is selected from Starch Glycolate Sodium (USP/NF/EP), corn starch, potato starch, tapioca starch, croscarmellose sodium, pre-gelatinized starch, sodium carboxymethylcellulose, alginates, resins, aqueous aluminum silicates, cross-linked polyvinylpyrrolidone, or any combination thereof.

In some embodiments, the surfactant is selected from Poloxamer Microprilled 188 (USP/NF/EP/JPE), sodium lauryl sulfate, polysorbates, or any combination thereof.

In some embodiments, the lubricant is selected from Magnesium Stearate 5712 (USP/NF/EP), stearic acid, glyceryl behenate, talc, carnauba wax, polymers of ethylene oxide, sodium lauryl sulfate, magnesium lauryl sulfate, sodium oleate, sodium stearyl fumarate, colloidal silicon dioxide, hydroxypropylmethylcellulose, hydroxypropylcellulose, acrylate-methacrylate copolymers, or any combination thereof.

In some embodiments, the color coating is selected from Opadry II White 85F18422 (Mfg. Std.), riboflavin, anthocyanin, paprika oleoresin, beet root, curcumin, indigo carmine, tartrazine, allura red, quinoline yellow; titanium dioxide, iron oxide, mica, betacarotene, or any combination thereof.

In some embodiments, the pharmaceutical composition includes vapendavir or a pharmaceutically acceptable salt thereof as described herein and further includes Cellulose Microcrystalline Silicified (PROSOLVHD90) (USP/NF), Starch Glycolate Sodium (USP/NF/EP), Poloxamer Microprilled 188 (USP/NF/EP/JPE), Magnesium Stearate 5712 (USP/NF/EP), and Opadry II White 85F18422 (Mfg. Std.).

The pharmaceutical composition or unit dosage form of the disclosure may be administered according to a dosage and administration regimen defined by routine testing in the light of the guidelines given above in order to obtain optimal activity while minim/zing toxicity or side effects for a particular subject. However, such fine tuning of the therapeutic regimen is routine in the light of the guidelines given herein.

The dosage of the compounds of the disclosure may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a subject will vary depending on the state and severity of the disorder and the physical condition of the subject. A measurable amelioration of any symptom or parameter can be determined by a person skilled in the art or reported by the subject to the physician. It will be understood that any clinically or statistically significant attenuation or amelioration of any symptom or parameter of urinary tract disorders is within the scope of the disclosure. Clinically significant attenuation or amelioration means perceptible to the subject and/or to the physician. It will be understood that the pharmaceutical formulations of the disclosure need not necessarily contain the entire amount of the compound that is effective in treating the disorder, as such effective amounts can be reached by administration of a plurality of divided doses of such pharmaceutical formulations.

In some embodiments, the pharmaceutical composition includes a diluent, such as Cellulose Microcrystalline Silicified (PROSOLVHD90) (USP/NF). In some embodiments, the pharmaceutical composition includes about 250 mg of a diluent, such as Cellulose Microcrystalline Silicified (PROSOLVHD90) (USP/NF). For example, the pharmaceutical composition may include about 200 mg, about 210 mg, about 220 mg, about 230 mg, about 240) mg, about 250 mg, about 260 mg, about 270 mg, about 280 mg, about 290 mg, about 300 mg of the diluent, or any range or value contained therein. In some embodiments, the pharmaceutical composition includes about 500 mg of a diluent, such as Cellulose Microcrystalline Silicified (PROSOLVHD90) (USP/NF). For example, the pharmaceutical composition may include about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570) mg, about 580 mg, about 590 mg, about 600 mg of the diluent, or any range or value contained therein, of the diluent, or any range or value contained therein.

In some embodiments, the pharmaceutical composition includes a disintegrant, such as Starch Glycolate Sodium (USP/NF/EP). In some embodiments, the pharmaceutical composition includes about 50 mg of a disintegrant, such as Starch Glycolate Sodium (USP/NF/EP). For example, the pharmaceutical composition may include about 40 mg, about 45 mg, about 50 mg, about 55 mg, about 60 of the disintegrant, or any range or value contained therein. In some embodiments, the pharmaceutical composition includes about 100 mg of a disintegrant, such as Starch Glycolate Sodium (USP/NF/EP). For example, the pharmaceutical composition may include about 80 mg, about 90 mg, about 100 mg, about 110 mg, about 120 of the disintegrant, or any range or value contained therein.

In some embodiments, the pharmaceutical composition includes a surfactant, such as Poloxamer Microprilled 188 (USP/NF/EP/JPE). In some embodiments, the pharmaceutical composition includes about 10 mg of a surfactant, such as Poloxamer Microprilled 188 (USP/NF/EP/JPE). For example, the pharmaceutical composition may include about 8 mg, about 9 mg, about 10 mg, about 11 mg, about 12 mg of the surfactant, or any range or value contained therein. In some embodiments, the pharmaceutical composition includes about 20 mg of a surfactant, such as Poloxamer Microprilled 188 (USP/NF/EP/JPE). For example, the pharmaceutical composition may include about 16 mg, about 18 mg, about 20 mg, about 22 mg, about 24 mg of the surfactant, or any range or value contained therein.

In some embodiments, the pharmaceutical composition includes a lubricant, such as Magnesium Stearate 5712 (USP/NF/EP). In some embodiments, the pharmaceutical composition includes about 10 mg of a lubricant, such as Magnesium Stearate 5712 (USP/NF/EP). For example, the pharmaceutical composition may include about 8 mg, about 9 mg, about 10 mg, about 11 mg, about 12 mg of the lubricant, or any range or value contained therein. In some embodiments, the pharmaceutical composition includes about 20 mg of a lubricant, such as Magnesium Stearate 5712 (USP/NF/EP). For example, the pharmaceutical composition may include about 16 mg, about 18 mg, about 20 mg, about 22 mg, about 24 mg of the lubricant, or any range or value contained therein.

In some embodiments, the pharmaceutical composition includes a color coating, such as Opadry II White 85F18422 (Mfg. Std.). In some embodiments, the pharmaceutical composition includes about 15 mg of a color coating, such as Opadry II White 85F18422 (Mfg. Std.). For example, the pharmaceutical composition may include about 12 mg, about 13 mg, about 14 mg, about 15 mg, about 16 mg, about 17 mg, about 18 mg of the color coating, or any range or value contained therein. In some embodiments, the pharmaceutical composition includes about 15 mg of a color coating, such as Opadry II White 85F18422 (Mfg. Std.). For example, the pharmaceutical composition may include about 24 mg, about 26 mg, about 28 mg, about 30 mg, about 32 mg, about 34 mg, about 36 mg of the color coating, or any range or value contained therein.

In some embodiments, the pharmaceutical composition includes about 250 mg of vapendavir or a pharmaceutically acceptable salt thereof, about 250 mg of Cellulose Microcrystalline Silicified (PROSOLVHD90) (USP/NF), about 50 mg of Starch Glycolate Sodium (USP/NF/EP), about 10 mg of Poloxamer Microprilled 188 (USP/NF/EP/JPE), about 10 mg of Magnesium Stearate 5712 (USP/NF/EP), and about 15 mg of Opadry II White 85F18422 (Mfg. Std.).

In some embodiments, the pharmaceutical composition includes about 500 mg of vapendavir or a pharmaceutically acceptable salt thereof, about 500 mg of Cellulose Microcrystalline Silicified (PROSOLVHD90) (USP/NF), about 100 mg of Starch Glycolate Sodium (USP/NF/EP), about 20 mg of Poloxamer Microprilled 188 (USP/NF/EP/JPE), about 20 mg of Magnesium Stearate 5712 (USP/NF/EP), and about 30 mg of Opadry II White 85F18422 (Mfg. Std.).

Some embodiments are directed to methods of treating a respiratory enterovirus infection in a human subject with comprising orally administering to the subject that is in a fed state, a therapeutically effective amount of vapendavir or a pharmaceutically acceptable salt thereof, thereby treating the respiratory enterovirus infection in the subject: wherein the therapeutically effective amount of vapendavir or a pharmaceutically acceptable salt thereof is between about 250 mg and about 2,000 mg per day: wherein the therapeutically effective amount of vapendavir or a pharmaceutically acceptable salt thereof is a single loading dose of about 1,000 mg followed by a maintenance dose of about 500 mg every 12 hours for about 4 to about 10 days, about 5 to about 8 days, about 6 to about 8 days or about 7 days: wherein treating the respiratory enterovirus infection in the subject comprises an improvement in the subjects' peak total lower respiratory symptom score (LRSS), an improvement in the signs and symptoms of the respiratory enterovirus infection, a decrease in respiratory enterovirus virus load and secondary bacterial infection frequency and severity, an improvement in pulmonary function tests (PFTs), or any combination thereof; and wherein the subject is not taking any medications that are known to be impacted by or significantly metabolized by CYP3A4. Preferably, the therapeutically effective amount of vapendavir or a pharmaceutically acceptable salt thereof is a single loading dose of about 1,000 mg followed a maintenance dose of about 500 mg every 12 hours for a total of about 13 maintenance doses.

The methods and compositions of the present disclosure may be employed in the following embodiments, which may be combined as appropriate to form new embodiments.
1. A method of treating a respiratory enterovirus infection in a human subject in need thereof, comprising orally administering to the subject a therapeutically effective amount of vapendavir or a pharmaceutically acceptable salt thereof, thereby treating the respiratory enterovirus infection in the subject.
2. The method of embodiment 1, wherein the therapeutically effective amount of vapendavir or a pharmaceutically acceptable salt thereof is between about 250 mg and about 2,000 mg per day.
3. The method of any of embodiments 1-2, wherein the respiratory enterovirus infection is selected from a rhinovirus, echovirus, EV-68, EV-71, coxsackie virus, a non-polio enterovirus and combinations thereof.
4. The method of any of embodiments 1-3, wherein the therapeutically effective amount of vapendavir or a pharmaceutically acceptable salt thereof is a single loading dose of about 1,000 mg followed by a maintenance dose of about 500 mg every 12 hours for about 4 to about 10 days.
5. The method of any of embodiments 1-4, wherein the therapeutically effective amount of vapendavir or a pharmaceutically acceptable salt thereof is a single loading dose of about 1,000 mg followed a maintenance dose of about 500 mg every 12 hours for a total of about 13 maintenance doses.
6. The method of any of embodiments 1-5, wherein the therapeutically effective amount of vapendavir or a pharmaceutically acceptable salt thereof is an amount that achieves a Cₘₐₓ of about 2000 to about 12000 ng/ml.
7. The method of any of embodiments 1-6, wherein the single loading dose is administered within about 48 hours of the subject displaying symptoms of a respiratory enterovirus infection.
8. The method of any of embodiments 1-7, wherein the single loading dose is administered within about 72 to about 96 hours of the subject displaying symptoms of a respiratory enterovirus infection.
9. The method of any of embodiments 1-8, wherein the symptoms of a respiratory enterovirus infection are selected from a cough, increased mucus production, sneezing, nasal congestion, runny nose, sore throat, headaches, muscle aches, breathlessness, tight chest, wheezing, fever, red eyes, swelling of the lymph nodes, fatigue, hoarse voice, rhinorrhea, malaise, nasal obstruction, sputum production, inability to function at normal level of activity, and any combinations thereof.
10. The method of any of embodiments 1-9, wherein the single loading dose is administered prior to the subject achieving a peak viral load.
11. The method of any of embodiments 1-10, wherein the subject is asymptomatic but has been diagnosed with a respiratory enterovirus infection.
12. The method of any of embodiments 1-11, wherein the maintenance dose is first administered about 6 to about 12 hours following administering the single loading dose.
13. The method of any of embodiments 1-12, wherein the single loading dose is administered after the subject has been diagnosed with a respiratory enterovirus infection, wherein the diagnosis is achieved by a clinical diagnosis, multiplex PCR testing, rapid antigen testing, direct fluorescent antibody testing, arterial blood gas testing, chest x-rays, peak flow meter testing, pleural fluid cultures, lung CT scanning, lung function tests, pulse oximetry testing, spirometry testing, sputum testing, nasal swab testing, throat swab testing, or any combination thereof.
14. The method of any of embodiments 1-13, wherein the single loading dose and maintenance dose are administered within about 30 minutes of the subject consuming a meal with solid food (e.g, the subject is in a fed state).
15. The method of any of embodiments 1-14, wherein administering the single loading dose and maintenance dose to a subject within about 30 minutes of the subject consuming a meal with solid food results in a higher plasma concentration of vapendavir or a pharmaceutically acceptable salt thereof than administering the single loading dose and maintenance dose to a subject in a fasted state.
16. The method of any of embodiments 1-15, wherein the therapeutically effective amount of vapendavir or a pharmaceutically acceptable salt thereof is a dose of about 500 mg every 12 hours for about 4 to about 10 days.
17. The method of any of embodiments 1-16, wherein a first dose is administered within about 48 hours of the subject displaying symptoms of a respiratory enterovirus infection.
18. The method of any of embodiments 1-17, wherein a first dose is administered within about 72 to about 96 hours of the subject displaying symptoms of a respiratory enterovirus infection.
19. The method of any of embodiments 1-18, wherein the symptoms of a respiratory enterovirus infection are selected from a cough, increased mucus production, sneezing, nasal congestion, runny nose, sore throat, headaches, muscle aches, breathlessness, tight chest, wheezing, fever, red eyes, swelling of the lymph nodes, fatigue, hoarse voice, rhinorrhea, malaise, nasal obstruction, sputum production, inability to function at normal level of activity, and any combinations thereof.
20. The method of any of embodiments 1-19, wherein a first dose is administered prior to the subject achieving a peak viral load.
21. The method of any of embodiments 1-20, wherein the subject is asymptomatic but has been diagnosed with a respiratory enterovirus infection.
22. The method of any of embodiments 1-21, wherein the subject is a human under the age of about 6 years.
23. The method of any of embodiments 1-22, wherein a first dose is administered after the subject has been diagnosed with a respiratory enterovirus infection, wherein the diagnosis is achieved by a clinical diagnosis, multiplex PCR testing, rapid antigen testing, direct fluorescent antibody testing, arterial blood gas testing, chest x-rays, peak flow meter testing, pleural fluid cultures, lung CT scanning, lung function tests, pulse oximetry testing, spirometry testing, sputum testing, nasal swab testing, throat swab testing, or any combination thereof.
24. The method of any of embodiments 1-23, wherein the dose is administered within about 30 minutes of the subject consuming a meal with solid food (e.g, the subject is in a fed state).
25. The method of any of embodiments 1-24, wherein administering the dose to a subject within about 30 minutes of the subject consuming a meal with solid food results in a higher plasma concentration of vapendavir or a pharmaceutically acceptable salt thereof than administering the single loading dose and maintenance dose to a subject in a fasted state.
26. The method of any of embodiments 1-25, wherein the subject has an underlying condition selected from chronic obstructive pulmonary diseases (COPD), asthma, mixed COPD and asthma: cystic fibrosis (CF): bronchiolitis obliterans: fibrosis such as tuberculosis (TB), idiopathic pulmonary fibrosis (IPF), other major respiratory diagnosis (e.g., pneumonia, aspergillosis), non-CF bronchiectasis, COPD due to alpha-1 antitrypsin deficiency, active allergic rhinitis, nasal disease, (e.g. nasal polyposis, significant septal deviation, chronic rhinosinusitis, etc.); primary ciliary dyskinesia: bronchitis: pulmonary hypertension; pulmonary edema: acute respiratory distress syndrome (ARDS): pneumoconiosis: interstitial lung disease: mesothelioma: sickle cell disease, cancer, or any combination thereof.
27. The method of any of embodiments 1-26, wherein the subject is in a fed state.
28. The method of any of embodiments 1-27, wherein the subject is a human between about 40 and about 85 years of age.
29. The method of any of embodiments 1-28, wherein the subject been diagnosed with GOLD stage 2 COPD, GOLD stage 3 COPD, or GOLD stage 4 COPD.
30. The method of any of embodiments 1-29, wherein the subject is receiving stable maintenance therapy for COPD.
31. The method of any of embodiments 1-30, wherein treating the respiratory enterovirus infection in the subject comprises decreasing a viral load in the sputum, nasal passages, or a combination thereof in the subject compared with a viral load in the sputum, nasal passages, or a combination thereof of the subject prior to treatment.
32. The method of any of embodiments 1-31, wherein treating the respiratory enterovirus infection in the subject comprises decreasing, and/or preventing an increase in the subjects' Evaluating Respiratory Symptoms in COPD Score (E-RS) compared to the subjects' Evaluating Respiratory Symptoms in COPD Score (E-RS) prior to treatment.
33. The method of any of embodiments 1-32, wherein treating the respiratory enterovirus infection in the subject comprises decreasing, and/or preventing an increase in the subjects' peak lower respiratory symptom score (LRSS) compared to the subjects' peak lower respiratory symptom score (LRSS) prior to treatment.
34. The method of any of embodiments 1-33, wherein treating the respiratory enterovirus infection in the subject comprises decreasing, and/or preventing an increase in the subjects' peak upper respiratory symptom score (URSS) compared to the subjects' peak upper respiratory symptom score (URSS) prior to treatment.
35. The method of any of embodiments 1-34, wherein treating the respiratory enterovirus infection in the subject comprises decreasing, and/or preventing an increase in the subjects' EXAcerbation of Chronic Pulmonary Disease Tool-Patient Reported Outcome (EXACT-PRO) compared to the subjects' EXAcerbation of Chronic Pulmonary Disease Tool-Patient Reported Outcome (EXACT-PRO) prior to treatment.
36. The method of any of embodiments 1-35, wherein treating the respiratory enterovirus infection in the subject comprises decreasing, and/or preventing an increase in the subjects' EXAcerbation of Chronic Pulmonary Disease Tool-Patient Respiratory Symptoms (EXACT-RS) compared to the subjects' EXAcerbation of Chronic Pulmonary Disease Tool-Patient Respiratory Symptoms (EXACT-RS) prior to treatment.
37. The method of any of embodiments 1-36, wherein treating the respiratory enterovirus infection in the subject comprises decreasing, and/or preventing an increase in the subjects' COPD assessment tool (CAT) compared to the subjects' COPD assessment tool (CAT) prior to treatment.
38. The method of any of embodiments 1-37, wherein treating the respiratory enterovirus infection in the subject comprises decreasing, and/or preventing an increase in the subjects' St. George's Respiratory Questionnaire (SGRQ) compared to the subjects' St. George's Respiratory Questionnaire (SGRQ) prior to treatment.
39. The method of any of embodiments 1-38, wherein treating the respiratory enterovirus infection in the subject comprises decreasing, and/or preventing an increase in the subjects' Wisconsin Upper Respiratory Symptom Survey (WURSS) compared to the subjects' Wisconsin Upper Respiratory Symptom Survey (WURSS) prior to treatment.
40. The method of any of embodiments 1-39, wherein treating the respiratory enterovirus infection in the subject comprises improving the subject's lung function compared to the subjects' lung function prior to treatment, and/or preventing a decrease in the subject's lung function compared to the subjects' lung function prior to treatment.
41. The method of any of embodiments 1-40, wherein the subjects' lung function is measured by Forced Expiratory Volume-1 (FEV₋₁: in liters and % predicted), Forced Vital Capacity (FVC: in liters and % predicted) FEV₁/FVC ratio and peak expiratory flow (PEF), or any combination thereof.
42. The method of any of embodiments 1-41, wherein treating the respiratory enterovirus infection in the subject comprises reducing, and/or preventing an increase in the subjects' peak nasal lavage viral load compared to the subjects' peak nasal lavage viral load prior to treatment.
43. The method of any of embodiments 1-42, wherein treating the respiratory enterovirus infection in the subject comprises reducing, and/or preventing an increase in the subjects' peak sputum lavage viral load compared to the subjects' peak sputum lavage viral load prior to treatment.
44. The method of any of embodiments 1-43, wherein treating the respiratory enterovirus infection in the subject comprises reducing, and/or preventing an increase in the subjects' AUC nasal viral load compared to the subjects' AUC nasal viral load prior to treatment.
45. The method of any of embodiments 1-44, wherein treating the respiratory enterovirus infection in the subject comprises reducing, and/or preventing an increase in the subjects' AUC sputum viral load compared to the subjects' AUC sputum viral load prior to treatment.
46. The method of any of embodiments 1-45, wherein treating the respiratory enterovirus infection in the subject comprises reducing the duration of viral shedding.
47. The method of any of embodiments 1-46, wherein treating the respiratory enterovirus infection in the subject comprises reducing, and/or preventing an increase in the subjects' peak sputum bacterial load compared to the subjects' peak sputum lavage bacterial load prior to treatment.
48. The method of any of embodiments 1-47, wherein treating the respiratory enterovirus infection in the subject comprises reducing, and/or preventing an increase in the subjects' AUC sputum bacterial load compared to the subjects' AUC sputum bacterial load prior to treatment.
49. The method of any of embodiments 1-48, wherein treating the respiratory enterovirus infection in the subject comprises reducing the number of days wherein the subject is positive for bacteria in the sputum.
50. The method of any of embodiments 1-49, wherein treating the respiratory enterovirus infection in the subject comprises preventing, or reducing, acute exacerbations of COPD in the subject caused by the respiratory enterovirus infection.
58. The method of any of embodiments 1-50, wherein treating the respiratory enterovirus infection in the subject comprises a reduction, and/or preventing an increase in in the frequency of acute exacerbations of the underlying condition in the subject, a reduction in the severity of acute exacerbations of the underlying condition in the subject, a reduction in the duration of acute exacerbations of the underlying condition in the subject or combinations thereof, caused by the respiratory enterovirus infection.
51. The method of any of embodiments 1-51, wherein administering to the subject a therapeutically effective amount of vapendavir or a pharmaceutically acceptable salt thereof exceeds the paEC₅₀ for human rhinoviruses.
52. The method of embodiment 1-52, wherein the paEC₅₀ for human rhinoviruses is about 75.4 ng/ml.
53. The method of any of embodiments 1-53, wherein the therapeutically effective amount of vapendavir or a pharmaceutically acceptable salt thereof is a single loading dose of about 1,000 mg followed by a maintenance dose of about 500 mg every 12 hours for about 4 to about 10 days.
54. The method of any of embodiments 1-54, wherein the therapeutically effective amount of vapendavir or a pharmaceutically acceptable salt thereof is a single loading dose of about 1,000 mg followed a maintenance dose of about 500 mg every 12 hours for a total of about 13 maintenance doses.
55. The method of any of embodiments 1-55, wherein the therapeutically effective amount of vapendavir or a pharmaceutically acceptable salt thereof is an amount that achieves a Cₘₐₓ of about 2000 to about 12000 ng/ml.
56. The method of any of embodiments 1-56, wherein the paEC₅₀ for human rhinoviruses is exceeded for about 4 to about 10 days or until treatment is stopped.
57. The method of any of embodiments 1-57, wherein administering to the subject a therapeutically effective amount of vapendavir or a pharmaceutically acceptable salt thereof exceeds the paEC₉₀ for human rhinoviruses.
58. The method of any of embodiments 1-58, wherein the paEC₉₀ for human rhinoviruses is about 678.6 ng/ml.
59. The method of any of embodiments 1-59, wherein the therapeutically effective amount of vapendavir or a pharmaceutically acceptable salt thereof is a single loading dose of about 1,000 mg followed by a maintenance dose of about 500 mg every 12 hours for about 4 to about 10 days.
60. The method of any of embodiments 1-60, wherein the therapeutically effective amount of vapendavir or a pharmaceutically acceptable salt thereof is a single loading dose of about 1,000 mg followed a maintenance dose of about 500 mg every 12 hours for a total of about 13 maintenance doses.
61. The method of any of embodiments 1-61, wherein the therapeutically effective amount of vapendavir or a pharmaceutically acceptable salt thereof is an amount that achieves a Cₘₐₓ of about 2000 to about 12000 ng/ml.
62. The method of any of embodiments 1-62, wherein the therapeutically effective amount of vapendavir or a pharmaceutically acceptable salt thereof is a single loading dose of about 1,000 mg followed by a maintenance dose of about 500 mg per day for about 6 to about 10 days.
63. The method of any of embodiments 1-63, wherein the paEC₉₀ for human rhinoviruses is exceeded for about 4 to about 10 days or until treatment is stopped.
64. The method of any of embodiments 1-64, wherein administering to the subject a therapeutically effective amount of vapendavir or a pharmaceutically acceptable salt thereof results in a Cₘₐₓ that exceeds the paEC₅₀ for human rhinoviruses.
65. The method of any embodiments 1-65, wherein the paEC₅₀ for human rhinoviruses is about 75.4 ng/ml.
66. The method of any of embodiments 1-66, wherein the therapeutically effective amount of vapendavir or a pharmaceutically acceptable salt thereof is a single loading dose of about 1,000 mg followed a maintenance dose of about 500 mg every 12 hours for a total of about 13 maintenance doses.
67. The method of any of embodiments 1-67, wherein the therapeutically effective amount of vapendavir or a pharmaceutically acceptable salt thereof is an amount that achieves a Cₘₐₓ of about 2000 to about 12000 ng/ml.
68. The method of any of embodiments 1-68, wherein the therapeutically effective amount of vapendavir or a pharmaceutically acceptable salt thereof is a single loading dose of about 1,000 mg followed by a maintenance dose of about 500 mg per day for about 6 to about 10 days.
69. The method of any of embodiments 1-69, wherein the paEC₅₀ for human rhinoviruses is exceeded for about 4 to about 10 days or until treatment is stopped.
70. The method of any of embodiments 1-70, wherein administering to the subject a therapeutically effective amount of vapendavir or a pharmaceutically acceptable salt thereof results in a Cₘₐₓ that exceeds the paEC₉₀ for human rhinoviruses.
71. The method of any of embodiments 1-71, wherein the paEC₉₀ for human rhinoviruses is about 678.6 ng/ml.
72. The method of any of embodiments 1-72, wherein the therapeutically effective amount of vapendavir or a pharmaceutically acceptable salt thereof is a single loading dose of about 1,000 mg followed a maintenance dose of about 500 mg every 12 hours for a total of about 13 maintenance doses.
73. The method of any of embodiments 1-73, wherein the therapeutically effective amount of vapendavir or a pharmaceutically acceptable salt thereof is an amount that achieves a Cₘₐₓ of about 2000 to about 12000 ng/ml.
74. The method of any of embodiments 1-74, wherein the therapeutically effective amount of vapendavir or a pharmaceutically acceptable salt thereof is a single loading dose of about 1,000 mg followed by a maintenance dose of about 500 mg per day for about 6 to about 10 days.
75. The method of any of embodiments 1-75, wherein the paEC₉₀ for human rhinoviruses is exceeded for about 4 to about 10 days or until treatment is stopped.
76. The method of any of embodiments 1-76, wherein administering to the subject a therapeutically effective amount of vapendavir or a pharmaceutically acceptable salt thereof exceeds the paEC₅₀ for human rhinoviruses following a single dose.
77. The method of any of embodiment 1-77, wherein the paEC₅₀ for human rhinoviruses is about 75.4 ng/ml.
78. The method of any of embodiments 1-78, wherein the therapeutically effective amount of vapendavir or a pharmaceutically acceptable salt thereof is a single loading dose of about 1,000 mg followed a maintenance dose of about 500 mg every 12 hours for a total of about 13 maintenance doses.
79. The method of any of embodiments 1-79, wherein the therapeutically effective amount of vapendavir or a pharmaceutically acceptable salt thereof is an amount that achieves a Cₘₐₓ of about 2000 to about 12000 ng/ml.
80. The method of any of embodiments 1-80, wherein the therapeutically effective amount of vapendavir or a pharmaceutically acceptable salt thereof is a single loading dose of about 1,000 mg followed by a maintenance dose of about 500 mg per day for about 6 to about 10 days.
81. The method of any of embodiments 1-81, wherein the paEC₅₀ for human rhinoviruses is exceeded for about 4 to about 10 days or until treatment is stopped.
82. The method of any of embodiments 1-82, wherein administering to the subject a therapeutically effective amount of vapendavir or a pharmaceutically acceptable salt thereof exceeds the paEC₉₀ for human rhinoviruses.
83. The method of any of embodiments 1-83, wherein the paEC₉₀ for human rhinoviruses is about 678.6 ng/ml.
84. The method of any of embodiments 1-84, wherein the therapeutically effective amount of vapendavir or a pharmaceutically acceptable salt thereof is a single loading dose of about 1,000 mg followed a maintenance dose of about 500 mg every 12 hours for a total of about 13 maintenance doses.
85. The method of any of embodiments 1-85, wherein the therapeutically effective amount of vapendavir or a pharmaceutically acceptable salt thereof is an amount that achieves a Cₘₐₓ of about 2000 to about 12000 ng/ml.
86. The method of any of embodiments 1-86, wherein the therapeutically effective amount of vapendavir or a pharmaceutically acceptable salt thereof is a single loading dose of about 1,000 mg followed by a maintenance dose of about 500 mg per day for about 6 to about 10 days.
87. The method of any of embodiments 1-87, wherein the paEC₉₀ for human rhinoviruses is exceeded for about 4 to about 10 days or until treatment is stopped.
88. The method of any of embodiments 1-88, wherein the therapeutically effective amount of vapendavir or a pharmaceutically acceptable salt thereof is in tablet form.
89. The method of any of embodiments 1-89, wherein the vapendavir is a free base, maleate salt, oxalate salt, phosphate salt, sulfate salt, tartrate salt, or chloride salt.
90. The method of any of embodiments 1-90, wherein the vapendavir is an amorphous form of vapendavir or a pharmaceutically acceptable salt thereof.
91. A pharmaceutical composition comprising a therapeutically effective amount of vapendavir or a pharmaceutically acceptable salt thereof.
92. The pharmaceutical composition of embodiment 91, wherein the vapendavir is a free base, maleate salt, oxalate salt, phosphate salt, sulfate salt, or chloride salt.
93. The pharmaceutical composition of any of embodiments 91-92, wherein the vapendavir is an amorphous form of vapendavir or a pharmaceutically acceptable salt thereof.
94. The pharmaceutical composition of any of embodiments 91-93, comprising about 250 mg of vapendavir or a pharmaceutically acceptable salt thereof.
95. The pharmaceutical composition of any of embodiments 91-94, comprising about 500 mg of vapendavir or a pharmaceutically acceptable salt thereof.
96. The pharmaceutical composition of any of embodiments 91-95, wherein the pharmaceutical composition is a tablet.
97. The pharmaceutical composition of any of embodiments 91-96, further comprising a diluent, a disintegrant, a surfactant, a lubricant, a color coating, or any combination thereof.
98. The pharmaceutical composition of any of embodiments 91-97, wherein the diluent is selected from Cellulose Microcrystalline Silicified (PROSOLVHD90) (USP/NF), anhydrous calcium phosphate, anhydrous lactose, calcium carbonate, calcium lactate, calcium sulfate dihydrate, corn starch, fructose, kaolin, lactose monohydrate, magnesium hydroxide, maltitol, maltose monohydrate, mannitol, sorbitol, sucrose, tribasic calcium phosphate, sodium citrate, glycine, croscarmellose sodium, ethanol, propylene glycol, glycerin, polyvinylpyrrolidone, hydroxypropylmethylcellulose (HPMC), hydroxypropylcellulose (HPC), sucrose, gelatin, acacia, or any combination thereof.
99. The pharmaceutical composition of any of embodiments 91-98, wherein the disintegrant is selected from Starch Glycolate Sodium (USP/NF/EP), corn starch, potato starch, tapioca starch, croscarmellose sodium, pre-gelatinized starch, sodium carboxymethylcellulose, alginates, resins, aqueous aluminum silicates, cross-linked polyvinylpyrrolidone, or any combination thereof.
100. The pharmaceutical composition of any of embodiments 91-99, wherein the surfactant is selected from Poloxamer Microprilled 188 (USP/NF/EP/JPE), sodium lauryl sulfate, polysorbates, or any combination thereof.
101. The pharmaceutical composition of any of embodiments 91-100, wherein the lubricant is selected from Magnesium Stearate 5712 (USP/NF/EP), stearic acid, glyceryl behenate, talc, carnauba wax, polymers of ethylene oxide, sodium lauryl sulfate, magnesium lauryl sulfate, sodium oleate, sodium stearyl fumarate, colloidal silicon dioxide, hydroxypropylmethylcellulose, hydroxypropylcellulose, acrylate-methacrylate copolymers, or any combination thereof.
102. The pharmaceutical composition of any of embodiments 91-101, wherein the color coating is selected from Opadry II White 85F18422 (Mfg. Std.), riboflavin, anthocyanin, paprika oleoresin, beet root, curcumin, indigo carmine, tartrazine, allura red, quinoline yellow, titanium dioxide, iron oxide, mica, betacarotene, or any combination thereof.
103. The pharmaceutical composition of any of embodiments 91-102, wherein the pharmaceutical composition further comprises Cellulose Microcrystalline Silicified (PROSOLVHD90) (USP/NF), Starch Glycolate Sodium (USP/NF/EP), Poloxamer Microprilled 188 (USP/NF/EP/JPE), Magnesium Stearate 5712 (USP/NF/EP), and Opadry II White 85F18422 (Mfg. Std.).
104. The pharmaceutical composition of any of embodiments 91-103, wherein the pharmaceutical composition further comprises about 250 mg of Cellulose Microcrystalline Silicified (PROSOLVHD90) (USP/NF).
105. The pharmaceutical composition of any of embodiments 91-104, wherein the pharmaceutical composition further comprises about 500 mg of Cellulose Microcrystalline Silicified (PROSOLVHD90) (USP/NF).
106. The pharmaceutical composition of any of embodiments 91-105, wherein the pharmaceutical composition comprises about 50 mg of Starch Glycolate Sodium (USP/NF/EP).
107. The pharmaceutical composition of any of embodiments 91-106, wherein the pharmaceutical composition comprises about 100 mg of Starch Glycolate Sodium (USP/NF/EP).
108. The pharmaceutical composition of any of embodiments 91-107, wherein the pharmaceutical composition comprises about 10 mg of Poloxamer Microprilled 188 (USP/NF/EP/JPE).
109. The pharmaceutical composition of any of embodiments 91-108, wherein the pharmaceutical composition comprises about 20 mg of Poloxamer Microprilled 188 (USP/NF/EP/JPE).
110. The pharmaceutical composition of any of embodiments 91-109, wherein the pharmaceutical composition comprises about 10 mg of Magnesium Stearate 5712 (USP/NF/EP).
111. The pharmaceutical composition of any of embodiments 91-110, wherein the pharmaceutical composition comprises about 20 mg of Magnesium Stearate 5712 (USP/NF/EP).
112. The pharmaceutical composition of any of embodiments 91-111, wherein the pharmaceutical composition comprises about 15 mg of Opadry II White 85F18422 (Mfg. Std.).
113. The pharmaceutical composition of any of embodiments 91-112, wherein the pharmaceutical composition comprises about 30 mg of Opadry II White 85F18422 (Mfg. Std.).
114. The pharmaceutical composition of any of embodiments 91-113, wherein the pharmaceutical composition comprises about 250 mg of vapendavir or a pharmaceutically acceptable salt thereof, about 250 mg of Cellulose Microcrystalline Silicified (PROSOLVHD90) (USP/NF), about 50 mg of Starch Glycolate Sodium (USP/NF/EP), about 10 mg of Poloxamer Microprilled 188 (USP/NF/EP/JPE), about 10 mg of Magnesium Stearate 5712 (USP/NF/EP), and about 15 mg of Opadry II White 85F18422 (Mfg. Std.).
115. The pharmaceutical composition of any of embodiments 91-114, wherein the pharmaceutical composition comprises about 500 mg of vapendavir or a pharmaceutically acceptable salt thereof, about 500 mg of Cellulose Microcrystalline Silicified (PROSOLVHD90) (USP/NF), about 100 mg of Starch Glycolate Sodium (USP/NF/EP), about 20 mg of Poloxamer Microprilled 188 (USP/NF/EP/JPE), about 20 mg of Magnesium Stearate 5712 (USP/NF/EP), and about 30 mg of Opadry II White 85F18422 (Mfg. Std.).

### EXAMPLES

The following examples were or will be carried out according to embodiments of the present disclosure.

EXAMPLE 1—A double-blind, randomized, placebo-controlled trial in participants with chronic obstructive pulmonary disease (COPD) to evaluate the impact of vapendavir on the development of lower respiratory tract symptoms following rhinovirus challenge

Introduction: Rhinoviruses and COPD. RVs are small RNA viruses that are ubiquitous and thought to number >180 different strains. RVs can be divided into RV-A. RV—B and RV-C families based on sequence homology. While normally associated with mild, self-limiting illnesses in otherwise healthy individuals. RVs can cause significant morbidity and mortality via exacerbating established chronic respiratory diseases such as asthma. COPD or cystic fibrosis, causing complications in immunocompromised individuals such as transplant recipients, and precipitating moderate or severe illness in elderly people. Currently, there is no approved treatment or vaccine for RV infection, and very few RV specific antiviral drugs are in late-stage clinical development.

Importantly, approximately 40 to 50% of COPD exacerbations (range 10 to 70%) are linked to RV infection. The current therapies for managing COPD and treating acute exacerbations (e.g., long acting muscarinic receptor antagonists (LAMAs), long acting β2 receptor agonists (LABAS), inhaled corticosteroids (ICS), and antibacterial drugs) are again only partially effective, have significant side effects and often fail to address the underlying mechanisms of disease or triggers of exacerbation. Exacerbations are associated with increased airway inflammation: increased mucus production, delayed or deficient antiviral host defenses and increases in pathogenic respiratory bacteria.

The complex biology of COPD exacerbations has recently been illustrated using RV challenge models in patients with COPD. Mallia et al, demonstrated that RV infection induces the symptomatic, physiologic and inflammatory features reported in naturally occurring COPD exacerbations, and that infection is associated with impaired antiviral host factors and excessive neutrophilic inflammation. This study also highlighted the temporal relationship between symptoms, virus load and mechanisms of disease, with upper respiratory symptoms beginning shortly after viral challenge, and peak lower respiratory symptoms, neutrophil inflammation, and secondary bacterial infection following at time points >1 week from onset of initial symptoms. The use of the RV challenge model in COPD participants has therefore revealed a potential treatment window for intervention of acute COPD exacerbations associated with RVs by using potent specific antiviral treatments.

Dosing. The dose selected for this study is a 1.000 mg loading dose followed by daily dosing at 500 mg BID for 7 days. The second dose may be given on day 1 (12 hr later) if the loading dose is given in the morning. The 2nd dose on day 2 (12 h later) will be 500 mg, and Days 2 through 7 (only 1st dose given on Day 7) will be 500 mg BID (12 h apart).

Study Rationale. An effective, easily administered therapy for RV infection would have the potential to reduce the serious health effects in vulnerable populations, such as those with COPD. Treatment of RV infection could reduce the severity and frequency of acute COPD exacerbations, preserve pulmonary function, prevent secondary bacterial infections, and mitigate the need for costly medical interventions, including inhaled agents, steroids, antibacterial drugs, emergency treatment, hospitalization, and mechanical ventilation. Thus, early treatment of an RV infection could potentially improve both the quality of life and longevity of COPD patients.

### Objectives

To evaluate the effectiveness of vapendavir vs. placebo
To evaluate the safety and tolerability of vapendavir vs. placebo
To evaluate the PK/PD relationship between vapendavir exposures, viral load, and other outcomes of interest

Primary Endpoint. In participants with COPD, the primary endpoint will be evaluation of the peak total lower respiratory symptom score (LRSS) in participants administered with vapendavir relative to/versus (vs) placebo as determined by Mallia et al. from day of treatment commencement until Day 42.

Secondary Endpoints. In participants with COPD, the secondary endpoints are to:
Evaluate the impact of vapendavir vs placebo on signs and symptoms of RV infection.
Evaluate the impact of vapendavir vs placebo on RV virus load and secondary bacterial infection frequency and severity.
Evaluate the impact of vapendavir vs placebo on pulmonary function tests (PFTs).
Evaluate the safety and tolerability of vapendavir vs placebo.

Exploratory Endpoints. In participants with COPD, the exploratory endpoints are to:
Evaluate the PK/PD relationship between vapendavir exposure and virus load and other outcomes of interest.
Evaluate the impact of vapendavir vs placebo on medication changes (e.g., need for antibacterial drugs, rescue medications for pulmonary exacerbations).
Evaluate the impact of vapendavir vs placebo on utilization of health care resources (e.g., urgent care visits, hospitalization).
Evaluate the impact of vapendavir vs placebo on markers of inflammation/other biomarkers.
Evaluate the impact of vapendavir vs placebo on measures of molecular quantification of respiratory viruses and bacteria.

Overall Study Design. Randomization will be 1:1 vapendavir: placebo. Participants will undergo 7 days of twice daily treatment. Treatment will commence after symptom onset or when the participant determines they have a clinical cold. After 20±4 participants have completed 7 days of treatment and undergone a further 7 days of follow up; the DSMB and Sponsor will review the data from the first 20 participants (10±2 vapendavir: 10±2 placebo) in an interim analysis comprised of available safety and efficacy endpoints and make one of the following recommendations regarding the trial:
Continue with the study
Continue with the study, but with modifications
Terminate the study

The planned end of this clinical trial is defined as the last visit of the last participant.

### Participant Selection

Inclusion Criteria:
Male or female age ≥40 years and ≤75 years at the time of signing the informed consent form.
Confirmed diagnosis of Global Initiative for Chronic Obstructive Pulmonary Disease (GOLD) stage II COPD as defined by % predicted Forced expiratory volume in 1 second (FEV₁) ≥50% and FEV 1/Forced vital capacity (FVC)<70%.
History of acute exacerbations of COPD as defined by the participant answering “yes” to the question “do your COPD symptoms get noticeably worse when you catch a cold?”
If on maintenance therapy, be medically stable for at least 2 months prior to enrolment.
Clinically stable with no exacerbations within 2 months prior to enrolment.
Absence of other viral pathogens at baseline anterior nares sample by Genmark (Roche) molecular assay or similar respiratory panel.

Exclusion Criteria:
Participants with other causes of chronic airflow limitation:
Including but not limited to: Asthma (mixed COPD and asthma is acceptable); cystic fibrosis (CF); bronchiolitis obliterans; and fibrosis such as tuberculosis (TB), idiopathic pulmonary fibrosis (IPF), or other major respiratory diagnosis (e.g., pneumonia, aspergillosis), etc.
Non-CF bronchiectasis.
Any disorder, for example, cardiovascular, gastrointestinal, hepatic, renal, neurological, musculoskeletal, infectious, endocrine, metabolic, hematological, psychiatric impairment that is not medically stable, or other major physical impairment that is not considered by the investigator medically stable/controlled.
Medications that could be impacted by CYP3A4 induction and have serious complications for the participant within the treatment period without the ability to discontinue safely with a sufficient washout period before initiating vapendavir.
History of clinically significant infection (respiratory or non-respiratory) requiring antibiotic or systemic steroids >10 mg/day within 30 days prior to planned RV challenge.
Pregnant, planning to become pregnant or nursing females during and within 30 days of treatment.
Any cold symptom within the last 6 weeks such as sore throat, sneezing, rhinorrhea, malaise, nasal obstruction or cough.
Presence (at screening) of serum rhinovirus 16 neutralizing antibody titers at greater than or equal to one in four (≥1/4) dilution.
Active allergic rhinitis, active nasal disease such as nasal polyposis, chronic rhinosinusitis etc.
Active alcohol and/or drug misuse, at the discretion of the Investigator.
Use of any over the counter cold prophylaxis products including nasal sprays, C-vitamins, zinc or Echinacea or estrogen-based hormone replacement therapy within 1 month prior to the enrolment.
Participation in other clinical trial with medical investigational product within 30 days or 5 drug half-lives (whatever is longer) prior to enrolment.
Hypersensitivity/allergy to any of the active or placebo ingredients/components.
Patients with immunosuppression (e.g., human immunodeficiency virus (HIV), transplant recipients on anti-rejection medications, those undergoing chemo- or immuno-therapy).
Other factors that in the opinion of the investigator are considered a risk.

Study Integrity. This will be a double-blind study, placebo-controlled study.

Number of Participants. There will be up to 50 enrolled participants who will complete the study procedures (up to 25 per arm).

Lung Function. British Thoracic Society guidelines will be followed in the conduct of all lung function (spirometry) assessments. On the days of clinic visits, trained personnel utilizing calibrated equipment will conduct pulmonary function tests and measure FEV₁ (in liters and % predicted), FVC (in liters and % predicted) FEV₁/FVC ratio and PEF.

Intranasal RV-A16 Challenge. Participants will be challenged with 100 Tissue Culture Infective dose causing tissue culture infective dose causing 50% cytotoxicity (TCID50) of rhinovirus RV-A16 in a negative pressure chamber within ICRRU at the Imperial College Healthcare NHS Trust. This process will be performed as per the SOP or Site Operational Manual. RV-A16 will be diluted and using an atomizer (De Vilbiss Co.) and introduced into both nostrils by four inhalations. The participants will be encouraged to sit still without swallowing for several minutes to aid introduction of the challenge virus into the nasal passage.

Placebo. Placebo will be dispensed by the study team on Day 0 at the clinical visit at the study site. Participants will be given enough Placebo for the 7-day treatment phase. (e.g., Days 2 to 8). Treatment is twice daily, am and pm. 12 h apart #2 h. Placebo tablets will be matched to the active treatment and will be administered orally for 7 days as follows: 4 tablets for the initial dose as soon as symptoms are present or the subject answers “yes” to “do you think you have a cold” on the first day of dosing (e.g. Day 2), and 2 tablets, either on the same day (e.g., Day 2) or the next day. Treatment will therefore be for a total of 7 days (consisting of an initial 4-tablet dose followed by thirteen 2-tablet doses). Treatment may occur within the home, on non-clinical study days. All tablets to be swallowed with ample water and taken within 30 minutes of consuming a meal with solid food.

Vapendavir. Vapendavir will be dispensed by the study team on Day 0 at the clinical study visit at the study site. Participants will be given enough vapendavir for the 7-day treatment phase, (e.g., Days 2 to 8). Treatment is twice daily, am and pm. 12 h apart+2 h. vapendavir micronized free base tablets (250 mg each) will be given orally beginning with a 1,000 mg loading dose (4 tablets) as soon as symptoms are present or the subject answers “yes” to “do you think you have a cold” followed by 500 mg (2 tablets) either on the same day (e.g., day 2) or the next day. Treatment will therefore be for a total of 7 days consisting of an initial 1,000 mg dose (4-tablets) followed by thirteen 500 mg doses (2-tablets). Treatment may occur within the home, on non-clinical study days. All tablets to be swallowed with ample water and taken within 30 minutes of consuming a meal with solid food.

### Concomitant Medications, Treatments, and Procedures

Standard of care medications for the management of chronic COPD are allowed; examples are provided in TABLE 1. Oral steroids >10 mg/day are not allowed at screening/baseline.

TABLE 1

| Drug Class/Type | Examples |
|---|---|
| Short-acting bronchodilators | Albuterol (Proair HFA, Ventolin HFA) |
| | Levalbuterol (Xopenex) |
| | Ipratropium (Atrovent HFA) |
| | Albuterol/ipratropium (Combivent Respimat) |
| Long-acting bronchodilators | Aclidinium (Tudorza) |
| | Arformoterol (Brovana) |
| | Formoterol (Foradil, Perforomist) |
| | Glycopyrrolate (Seebri Neohaler, Lonhala Magnair) |
| | Indacaterol (Arcapta) |
| | Olodaterol (Striverdi Respimat) |
| | Revefenacin (Yupelri) |
| | Salmeterol (Serevent) |
| | Tiotropium (Spiriva) |
| | Umeclidinium (Incruse Ellipta) |
| Corticosteroids | Fluticasone (Flovent) |
| | Budesonide (Pulmicort) |
| | Prednisolone (<10 mg/day for chronic maintenance |
| | therapy; not for acute exacerbation) |
| | Beclometasone |
| LABA, LAMA and combination | Aclidinium/formoterol (Duaklir) |
| therapies: | |
| LABA and LAMA combination | Glycopyrrolate/formoterol (Bevespi Aerosphere) |
| bronchodilator therapies | Tiotropium/olodaterol (Stiolto Respimat) |
| | Umeclidinium/vilanterol (Anoro Ellipta) |
| | Glycopyrronium/indacaterol |
| Combinations of an ICS and a long- | Budesonide/formoterol (Symbicort) |
| acting bronchodilator | Fluticasone/salmeterol (Advair) |
| | Fluticasone/vilanterol (Breo Ellipta) |
| | Beclometasone/formoterol |
| Triple Combination Therapies | Fluticasone/umeclidinium/vilanterol |
| (ICS/LAMA/LABA) | Budesonide/glycopironium/formoterol |
| | Beclometasone/formoterol/glycopyrronium |
| Methylxanthines | Theophylline |
| Phosphodiesterase-4 inhibitor | Roflumilast (Daliresp) |
| Mucoactive drugs | Carbocysteine |
| | Erdosteine |
| | N-acetylcysteine |
| Biologics/anti-eosinophilia drugs | Mepolizumab (Nucala) |
| | Benralizumab (Fasenra) |
| | Reslizumab (Cinqair) |
| | Dupilumab (Dupixent |
| Biologics/anti-eosinophilia drugs | Mepolizumab (Nucala) |
| | Benralizumab (Fasenra) |
| | Reslizumab (Cinqair) |
| | Dupilumab (Dupixent) |
| Other prescribed for COPD | With Sponsor/designee approval |

### Statistical Methods

Primary Endpoint. The primary statistical endpoint is the difference in change from baseline peak total LRSS as determined by Mallia et al. from day of treatment commencement until Day 42. Total LRSS each day is calculated as the mean of the total LRSS scored in the am and pm each day. The total LRSS is composed of the sum of the individual symptom scores at each scoring. Peak is defined as the highest value of the total symptom scores on any day. Change from baseline will be calculated by subtracting the mean baseline total LRSS score from every other day (Day 0 to Day 42).

Secondary Endpoints. Some, but not all secondary objectives will be adequately powered with 50 participants per arm. The secondary objectives are:
1. Evaluate the impact of vapendavir vs placebo on signs and symptoms of RV infection. All will be calculated as a change from baseline:
   Difference in peak total URSS; day of treatment commencement to Day 42
   Difference in peak EXACT-RS; day of treatment commencement to Day 42
   Difference in peak EXACT-PRO; day of treatment commencement to Day 42
   Difference in AUC LRSS; day of treatment commencement to Day 42.
   Difference in AUC URSS; day of treatment commencement to Day 42
   Difference in AUC EXACT-RS; day of treatment commencement to Day 42
   Difference in AUC EXACT-PRO; day of treatment commencement to Day 42
   Number of days any URSS; day of treatment commencement to Day 42
   Number of days any LRSS; day of treatment commencement to Day 42.
   Difference in peak total EXACT-RS and EXACT-PRO score day of treatment commencement to Day 14
   Difference in peak total EXACT-RS and EXACT-PRO score day of treatment commencement to Day 28
   Difference in peak total EXACT-RS score day of treatment commencement to Day 42.
   Proportion of participants with change in total EXACT-RS ≥2 points from pre-dose to Day 7
   Proportion of participants with change in total EXACT-RS ≥2 points from pre-dose to Day 14
   Proportion of participants with change in total EXACT-RS ≥2 points from pre-dose to Day 28
   Mean change in AUC ER-S from pre-dose through 7 days of treatment and an additional 7 days of follow-up
   Mean change from baseline in to peak LRSS
   Difference in peak total EXACT-PRO score from pre-dose baseline to Day 7
   Difference in peak total EXACT-PRO score from pre-dose baseline to Day 14
   Difference in peak total SGRQ from pre-dose baseline to Day 28.
2. Evaluate the impact of vapendavir vs placebo on RV viral load and secondary bacterial infection
   Peak nasal lavage viral load (day of treatment commencement to Day 42).
   Peak sputum virus load (day of treatment commencement to Day 42).
   AUC nasal viral load (day of treatment commencement to Day 42).
   AUC sputum viral load (day of treatment commencement to Day 42).
   Duration of viral shedding (no. of days virus positive, Day 1 to Day 42)
   Proportion of participants positive for sputum bacteria (NHS microbiology, day of treatment commencement to Day 42).
   Peak sputum bacterial load (NHS microbiology, day of treatment commencement to Day 42).
   AUC sputum bacterial load (NHS microbiology, day of treatment commencement to Day 42).
   Number of days positive for bacteria (NHS microbiology, day of treatment commencement to Day 42).
3. Evaluate the impact of vapendavir vs placebo on lung function tests
   Maximum % change in each lung function test (FEV₁, FVC, FEV₁/FVC % predicted FEV₁, % predicted FVC) from baseline to after 7 days of treatment, and at least 7 additional days of follow-up after completing treatment.
   Difference in AUC of each lung function test through 7 days of treatment and at least 7 additional days of follow-up after completing treatment.
4. Evaluate the safety of vapendavir vs placebo
   Difference in any blood hematology test result on any day; day of treatment commencement to Day 42
   Difference in any LFTs result on any day; day of treatment commencement to Day 42
   Difference in any blood biochemistry result on any day; day of treatment commencement to Day 42
   Difference in no. of AEs or SAEs; day of treatment commencement to Day 42
   Difference in no. of participants reporting an AE or SAE; day of treatment commencement to Day 42
   Difference in no. of concomitant medications; day of treatment commencement to Day 42.
   Difference in any vital signs on any day; day of treatment commencement to Day 42
   Difference in the number of participants discontinuing due to safety or tolerability reasons

Exploratory Endpoint. The exploratory objectives are not all powered by 50 participants per arm. They will include:
1. Evaluate the relationship between vapendavir exposure and viral load and other outcomes of interest.
   Correlation of nasal lavage virus load with vapendavir PK-PD
   Correlation of sputum virus load with vapendavir PK-PD
2. Evaluate the impact of vapendavir vs placebo on medication changes in participants with COPD.
   % of participants requiring increases in the frequency and/or dose of their chronic COPD medications (e.g., LAMA, LABA) from day 1 through 28 days
   Changes in beta-agonist or anti-muscarinic agent inhaler or other respiratory medications (dose/frequency of use) from day 1 through 28 days
   % of participants requiring new treatment with systemic corticosteroids from day 1 through 28 days
   % of participants requiring new treatment with antibiotics from day 1 through 28 days
   % of participants requiring new treatment with both systemic corticosteroids and antibiotics from day 1 to 28 days
3. Evaluate the impact of vapendavir vs placebo on utilization of health care resources in participants with COPD.
   % of participants diagnosed with pneumonia from day of treatment commencement to Day 28%
   % of participants requiring ED evaluation
   % of participants requiring hospitalization
4. Evaluate the impact of vapendavir vs placebo on markers of inflammation/other biomarkers in participants with COPD.
   Peak serum CRP (day of treatment commencement to Day 42)
   AUC serum CRP (day of treatment commencement to Day 42).
   Peak nasal cytokine (each cytokine, day of treatment commencement to Day 42).
   AUC nasal cytokine (each cytokine, day of treatment commencement to Day 42).
   Peak sputum cytokine (each cytokine, day of treatment commencement to Day 42)
   AUC sputum cytokine (each cytokine, day of treatment commencement to Day 42)
   Peak total sputum cells (day of treatment commencement to Day 42)
   AUC total sputum cells (day of treatment commencement to Day 42).
   Peak any specific sputum cell subset (day of treatment commencement to Day 42).
   AUC any specific sputum cell subset (day of treatment commencement to Day 42)
5. Evaluate the impact of vapendavir vs placebo on measures of molecular quantification of microbes.
   Peak 16S rRNA values in sputum (day of treatment commencement to Day 42).
   AUC 16S rRNA values in sputum (day of treatment commencement to Day 42).
   Peak specific pathogenic bacteria load in sputum (qPCR, day of treatment commencement to Day 42)
   AUC specific pathogenic bacteria load in sputum (qPCR, day of treatment commencement to Day 42)
   No. of pathogens in sputum or nasal lavage (Genmark assay day of treatment commencement to Day 42)
   No. of participants positive for any pathogens in sputum or nasal lavage (Genmark assay day of treatment commencement to Day 42)
   Compare nasal lavage and sputum RV qPCR results with Genmark RV results.

### Example 2—Interim Pharmacokinetic Results from Normal Healthy Volunteers and Participants with a Diagnosis of COPD

### Group A—Normal Healthy Volunteer—(Period 1/Period 2—Single Dose Fast/Fed 500 mg: Period 3—MAD BID Dosing for 7 Days)

Group B—Participant with a Diagnosis of COPD (BID Dosing for 7 Days)

General Considerations: Normal Healthy Volunteers (Group A) received a 500 mg dose during Periods 1 and 2 (fast/fed part). Normal Healthy Volunteers (Group A) and participants with a diagnosis of COPD (Group B) received a loading dose of 1000 mg on the morning on Day 1 followed by a 500 mg pm dose and then received 500 mg BID up to Day 7. For calculation of Pharmacokinetic (PK) parameters, BLQ values were replaced by zero (0.00) before the first measurable concentration and by missing for any other time point. PK parameters related to the elimination phase are not reliable when RSQ-Adjusted was <0.80. Whenever it was the case, AUC_{0-inf}, Residual area, T_{1/2 el}, Cl/F, and V/F were not presented.

### Group A Concentration Data—Normal Healthy Volunteers (Group A-Periods 1 and 2 (Fast/Fed))

Table 2 shows descriptive statistics of vapendavir plasma concentrations vs time (Group A-Periods 1 and 2 (Fast/Fed)). Measurable concentrations are observed up to 72.0 hours post-dose for most subjects under fed conditions while measurable concentration are observed up to 48 hours post-dose for eight subjects, and 72 hours post-dose for 4 subjects under fasting conditions. Based on visual evaluation: Fed: Mean concentrations increased from pre-dose to 8 hours post-dose and decreased up to 72 h. Fast: Mean concentrations increased from pre-dose to 1 hour post-dose and decreased up to 72 h. Higher concentrations are observed following the administration under fed conditions when compared to the fast administration.

TABLE 2

| | | | | Relative Nominal Time (h) | | | | | | | | | | | | | |
|---|---|---|---|---|---|---|---|---|---|---|---|---|---|---|---|---|---|
| | | | | [symbol] | [symbol] | [symbol] | [symbol] | [symbol] | [symbol] | [symbol] | [symbol] | [symbol] | [symbol] | [symbol] | [symbol] | [symbol] | [symbol] |
| Group | Day | Treatment | Statistics | Concentration (mg/mL) | | | | | | | | | | | | | |
| A | 1 | Single dose of | N | [symbol] | [symbol] | [symbol] | [symbol] | [symbol] | [symbol] | [symbol] | [symbol] | [symbol] | [symbol] | [symbol] | [symbol] | [symbol] | [symbol] |
| | | 100 mg VPV, | | | | | | | | | | | | | | | |
| | | after a high- | | | | | | | | | | | | | | | |
| | | fat meal | | | | | | | | | | | | | | | |
| | | | Mean | [symbol] | [symbol] | [symbol] | [symbol] | [symbol] | [symbol] | [symbol] | [symbol] | [symbol] | [symbol] | [symbol] | [symbol] | [symbol] | [symbol] |
| | | | SD | [symbol] | [symbol] | [symbol] | [symbol] | [symbol] | [symbol] | [symbol] | [symbol] | [symbol] | [symbol] | [symbol] | [symbol] | [symbol] | [symbol] |
| | | | CV % | [symbol] | [symbol] | [symbol] | [symbol] | [symbol] | [symbol] | [symbol] | [symbol] | [symbol] | [symbol] | [symbol] | [symbol] | [symbol] | [symbol] |
| | | | Geometric | NC | NC | NC | [symbol] | [symbol] | [symbol] | [symbol] | [symbol] | [symbol] | [symbol] | [symbol] | [symbol] | [symbol] | [symbol] |
| | | | Mean | | | | | | | | | | | | | | |
| | | | Geometric | NC | NC | NC | [symbol] | [symbol] | [symbol] | [symbol] | [symbol] | [symbol] | [symbol] | [symbol] | [symbol] | [symbol] | [symbol] |
| | | | CV % | | | | | | | | | | | | | | |
| | | | | Relative Nominal Time (h) | | | | | | | | | | | | | |
| | | | | [symbol] | [symbol] | [symbol] | [symbol] | [symbol] | [symbol] | [symbol] | [symbol] | [symbol] | [symbol] | [symbol] | [symbol] | [symbol] | [symbol] |
| Group | Day | Treatment | Statistics | Concentration (mg/mL) | | | | | | | | | | | | | |
| A | 1 | Single dose of | N | [symbol] | [symbol] | [symbol] | [symbol] | [symbol] | [symbol] | [symbol] | [symbol] | [symbol] | [symbol] | [symbol] | [symbol] | [symbol] | [symbol] |
| | | 100 mg VPV, | | | | | | | | | | | | | | | |
| | | fasted | | | | | | | | | | | | | | | |
| | | | Mean | [symbol] | [symbol] | [symbol] | [symbol] | [symbol] | [symbol] | [symbol] | [symbol] | [symbol] | [symbol] | [symbol] | [symbol] | [symbol] | [symbol] |
| | | | SD | [symbol] | [symbol] | [symbol] | [symbol] | [symbol] | [symbol] | [symbol] | [symbol] | [symbol] | [symbol] | [symbol] | [symbol] | [symbol] | [symbol] |
| | | | CV % | NC | [symbol] | [symbol] | [symbol] | [symbol] | [symbol] | [symbol] | [symbol] | [symbol] | [symbol] | [symbol] | [symbol] | [symbol] | [symbol] |
| | | | Geometric | NC | NC | NC | [symbol] | [symbol] | [symbol] | [symbol] | [symbol] | [symbol] | [symbol] | [symbol] | [symbol] | [symbol] | [symbol] |
| | | | Mean | | | | | | | | | | | | | | |
| | | | Geometric | NC | NC | NC | [symbol] | [symbol] | [symbol] | [symbol] | [symbol] | [symbol] | [symbol] | [symbol] | [symbol] | [symbol] | [symbol] |
| | | | CV % | | | | | | | | | | | | | | |

[symbol]

indicates data missing or illegible when filed

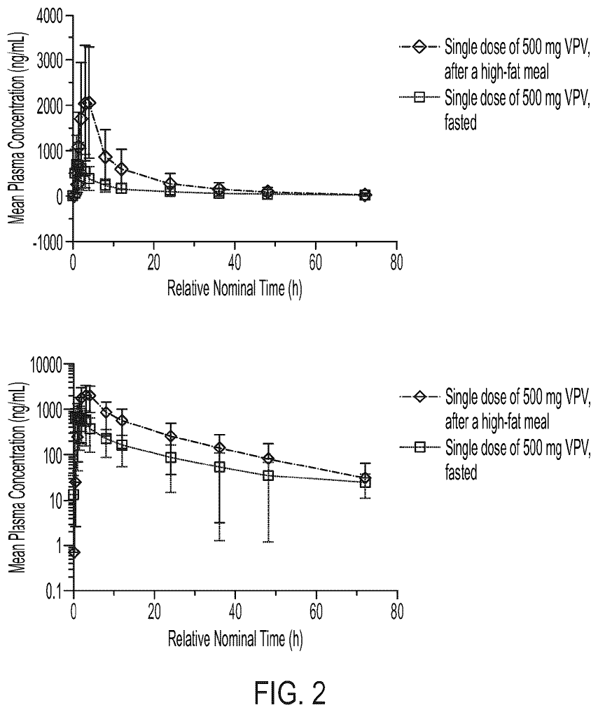

FIG. 2 depicts mean vapendavir plasma concentrations vs time profiles (Left=Linear Scale, Right=Log Scale) for Group A (Periods 1 and 2 (Fast/Fed).

Table 3 depicts descriptive statistics for PK parameters and discussion (Normal Healthy Volunteers (Group A-Periods 1 and 2 (Fast/Fed). The standard high-fat meal food intake prior to a single 500 mg VPV dose resulted in higher plasma concentrations for VPV relative to a single 500 mg dose under fasted conditions. The geometric LS mean ratio obtained are 325.36%, 329.51%, 316.11% and 274.03%. The intake of food highly increases the overall exposure of VPV. The mean peak concentration (Tₘₐₓ) was delay under fed conditions (3.06 h) when compared to fasting conditions (1.67 h). VPV plasma elimination half-lives were similar between fed and fasted dose groups. However, CL/F and V_{z}/F are higher under fast conditions.

TABLE 3

| | | | | AUC₀₋ₜ | AUC_{0-inf} | AUC₀₋₁₂ | AUC₀₋₂₄ | Cₘₐₓ | Residual |
|---|---|---|---|---|---|---|---|---|---|
| Group | Day | Treatment | Statistics | (h*ng/mL) | (h*ng/mL) | (h*ng/mL) | (h*ng/mL) | (ng/mL) | Area (%) |
| A | 1 | Single dose of | N | 8 | 8 | 8 | 8 | 8 | 8 |
| | | 500 mg VPV, | | | | | | | |
| | | after a high- | | | | | | | |
| | | fat meal | | | | | | | |
| | | | Mean | 23066.93 | 23694.22 | 13357.53 | 18188.56 | 2460 | 2.34 |
| | | | CV % | 69.30 | 70.54 | 58.55 | 62.86 | 50.47 | 62.41 |
| | | | Geometric | 18538.30 | 18984.95 | 11031.35 | 14821.28 | 2130 | 1.97 |
| | | | Mean | | | | | | |
| | | | Geometric | 83.04 | 83.24 | 81.09 | 82.81 | 67.67 | 70.81 |
| | | | CV % | | | | | | |

| | | | | Tₘₐₓ | T_{%} | K_{ct} | CL/F | Vz/F |
|---|---|---|---|---|---|---|---|---|
| Group | Day | Treatment | Statistics | (h) | (h) | (l/h) | (L/h) | (L) |
| A | 1 | Single dose of | N | 8 | 8 | 8 | 8 | 8 |
| | | 500 mg VPV, | | | | | | |
| | | after a high- | | | | | | |
| | | fat meal | | | | | | |
| | | | Mean | 3.06 | 13.47 | 0.0556 | 32.85 | 655.72 |
| | | | CV % | 30.78 | 24.49 | 36.09 | 70.11 | 81.47 |
| | | | Geometric | 2.91 | 13.03 | 0.0532 | 26.34 | 495.07 |
| | | | Mean | | | | | |
| | | | Geometric | 36.74 | 30.25 | 30.25 | 83.24 | 92.93 |
| | | | CV % | | | | | |

| | | | | AUC₀₋ₜ | AUC_{0-inf} | AUC₀₋₁₂ | AUC₀₋₂₄ | Cₘₐₓ | Residual |
|---|---|---|---|---|---|---|---|---|---|
| Group | Day | Treatment | Statistics | (h*ng/mL) | (h*ng/mL) | (h*ng/mL) | (h*ng/mL) | (ng/mL) | Area (%) |
| A | 1 | Single dose of | N | 9 | 9 | 9 | 9 | 9 | 9 |
| | | 500 mg VPV, | | | | | | | |
| | | fasted | | | | | | | |
| | | | Mean | 7289.78 | 7656.54 | 4021.14 | 5513.98 | 998 | 4.95 |
| | | | CV % | 72.81 | 73.42 | 61.29 | 63.65 | 56.15 | 48.65 |
| | | | Geometric | 5614.64 | 5908.53 | 3255.92 | 4461.49 | 777 | 4.35 |
| | | | Mean | | | | | | |
| | | | Geometric | 93.71 | 92.38 | 86.43 | 83.79 | 110.99 | 62.79 |
| | | | CV % | | | | | | |

| | | | | Tₘₐₓ | T_{%} | K_{ct} | CL/F | Vz/F |
|---|---|---|---|---|---|---|---|---|
| Group | Day | Treatment | Statistics | (h) | (h) | (l/h) | (L/h) | (L) |
| A | 1 | Single dose of | N | 9 | 9 | 9 | 9 | 9 |
| | | 500 mg VPV, | | | | | | |
| | | fasted | | | | | | |
| | | | Mean | 1.67 | 13.53 | 0.0569 | 109.95 | 1942.30 |
| | | | CV % | 61.85 | 30.01 | 38.59 | 76.03 | 78.09 |
| | | | Geometric | 1.40 | 12.90 | 0.0537 | 84.62 | 1574.55 |
| | | | Mean | | | | | |
| | | | Geometric | 70.93 | 35.41 | 35.41 | 92.36 | 72.65 |
| | | | CV % | | | | | |

| | | Reference | Test | Ratio¹ |
|---|---|---|---|---|
| PK | | Geometric | Geometric | Fed/Fast |
| Parameter | Units | LSM | LSM | (%) |
| AUC₀₋ₜ | h*ng/mL | 5614.64 | 18267.98 | 325.36 |
| AUC₀₋₂₄ | h*ng/mL | 4461.49 | 14700.89 | 329.51 |
| AUC_{0-inf} | h*ng/mL | 5908.53 | 18677.63 | 316.11 |
| Cₘₐₓ | ng/mL | 776.94 | 2129.02 | 274.03 |

| | | 90% | 90% |
|---|---|---|---|
| PK | | Lower | Upper |
| Parameter | Units | CI (%)² | CI (%)² |
| AUC₀₋ₜ | h*ng/mL | 173.52 | 610.07 |
| AUC₀₋₂₄ | h*ng/mL | 178.77 | 607.34 |
| AUC_{0-inf} | h*ng/mL | 169.98 | 587.88 |
| Cₘₐₓ | ng/mL | 135.19 | 555.42 |

Table 4 depicts descriptive statistics of plasma concentrations vs time (Group A-Treatment C (Days 1-7). Measurable concentrations are observed up to 12.0 hours post-dose on Day 1 for all subjects. The first measurable concentration was observed at 0.500 hours for most subjects. Measurable concentrations are observed from 0.00 up to 12.0 hours post-dose on Day 4. On Day 7, measurable concentrations are observed up to 48.0 hours post-dose (end of sampling schedule for Day 7) for all subjects. Based on visual evaluation: Lower concentrations are obtained on Day 1 when compared to Day 4 and 7. Lower concentrations are obtained on Day 4 when compared to Day 7.

TABLE 4

| | | | | Relative Nominal Time (h) | | | | |
|---|---|---|---|---|---|---|---|---|
| | | | | 0.00 | 0.250 | 0.500 | 1.00 | 1.50 |
| Group | Day | Treatment | Statistics | Concentration (ng/mL) | | | | |
| A | 1 | VPV 1000 | N | 8 | 8 | 8 | 8 | 8 |
| | | mg am and | | | | | | |
| | | 500 mg pm | | | | | | |
| | | | Mean | 0.00 | 1.59 | 215 | 988 | 1060 |
| | | | SD | 0.00 | 4.49 | 284 | 1210 | 1130 |
| | | | CV % | NC | 283 | 132 | 122 | 106 |
| | | | Geometric | NC | NC | NC | 418 | 588 |
| | | | Mean | | | | | |
| | | | Geometric | NC | NC | NC | 359 | 239 |
| | | | CV % | | | | | |

| | | | | Relative Nominal Time (h) | | | | |
|---|---|---|---|---|---|---|---|---|
| | | | | 2.00 | 3.00 | 4.00 | 8.00 | 12.0 |
| Group | Day | Treatment | Statistics | Concentration (ng/mL) | | | | |
| A | 1 | VPV 1000 | N | 8 | 8 | 8 | 8 | 8 |
| | | mg am and | | | | | | |
| | | 500 mg pm | | | | | | |
| | | | Mean | 977 | 705 | 554 | 277 | 215 |
| | | | SD | 724 | 466 | 360 | 171 | 143 |
| | | | CV % | 74.1 | 66.1 | 65.1 | 61.8 | 66.3 |
| | | | Geometric | 644 | 529 | 453 | 233 | 176 |
| | | | Mean | | | | | |
| | | | Geometric | 177 | 115 | 78.6 | 69.4 | 78.2 |
| | | | CV % | | | | | |

| | | | | Relative Nominal Time (h) | | | | |
|---|---|---|---|---|---|---|---|---|
| | | | | 0.00 | 0.250 | 0.500 | 1.00 | 1.50 |
| Group | Day | Treatment | Statistics | Concentration (ng/mL) | | | | |
| A | 4 | VPV 500 mg | N | 8 | 8 | 8 | 8 | 8 |
| | | BID | | | | | | |
| | | | Mean | 1270 | 1260 | 2080 | 2050 | 1770 |
| | | | SD | 422 | 414 | 1330 | 1150 | 820 |
| | | | CV % | 33.3 | 32.9 | 63.9 | 86.0 | 46.3 |
| | | | Geometric | 1190 | 1190 | 1750 | 1780 | 1580 |
| | | | Mean | | | | | |
| | | | Geometric | 41.0 | 38.5 | 72.3 | 70.8 | 58.8 |
| | | | CV % | | | | | |

| | | | | Relative Nominal Time (h) | | | | |
|---|---|---|---|---|---|---|---|---|
| | | | | 2.00 | 3.00 | 4.00 | 8.00 | 12.0 |
| Group | Day | Treatment | Statistics | Concentration (ng/mL) | | | | |
| A | 4 | VPV 500 mg | N | 8 | 8 | 8 | 8 | 8 |
| | | BID | | | | | | |
| | | | Mean | 1680 | 1530 | 1400 | 1080 | 832 |
| | | | SD | 715 | 631 | 518 | 442 | 410 |
| | | | CV % | 42.5 | 41.3 | 37.0 | 40.9 | 49.3 |
| | | | Geometric | 1540 | 1400 | 1310 | 996 | 747 |
| | | | Mean | | | | | |
| | | | Geometric | 48.8 | 50.2 | 44.0 | 47.2 | 53.8 |
| | | | CV % | | | | | |

| | | | | Relative Nominal Time (h) | | | | | |
|---|---|---|---|---|---|---|---|---|---|
| | | | | 0.00 | 0.250 | 0..500 | 1.00 | 1.50 | 2.00 |
| Group | Day | Treatment | Statistics | Concentration (ng/mL) | | | | | |
| A | 7 | VPV 500 mg | N | 7 | 7 | 7 | 7 | 7 | 7 |
| | | BID | | | | | | | |
| | | | Mean | 1770 | 1730 | 2490 | 2780 | 2430 | 2250 |
| | | | SD | 508 | 554 | 1370 | 1520 | 1120 | 868 |
| | | | CV % | 28.8 | 32.1 | 55.0 | 54.6 | 45.9 | 38.6 |
| | | | Geometric | 1700 | 1650 | 2180 | 2420 | 2190 | 2090 |
| | | | Mean | | | | | | |
| | | | Geometric | 31.5 | 34.3 | 60.2 | 63.4 | 56.1 | 43.7 |
| | | | CV % | | | | | | |

| | | | | Relative Nominal Time (h) | | | | | |
|---|---|---|---|---|---|---|---|---|---|
| | | | | 3.00 | 4.00 | 8.00 | 12.0 | 24.0 | 48.0 |
| Group | Day | Treatment | Statistics | Concentration (ng/mL) | | | | | |
| A | 7 | VPV 500 mg | N | 7 | 7 | 7 | 7 | 7 | 7 |
| | | BID | | | | | | | |
| | | | Mean | 2020 | 1880 | 1480 | 1120 | 671 | 233 |
| | | | SD | 723 | 747 | 805 | 554 | 410 | 196 |
| | | | CV % | 35.8 | 39.8 | 40.8 | 49.4 | 61.1 | 84.3 |
| | | | Geometric | 1910 | 1750 | 1370 | 1000 | 560 | 166 |
| | | | Mean | | | | | | |
| | | | Geometric | 38.2 | 41.8 | 45.9 | 55.1 | 75.7 | 118 |
| | | | CV % | | | | | | |

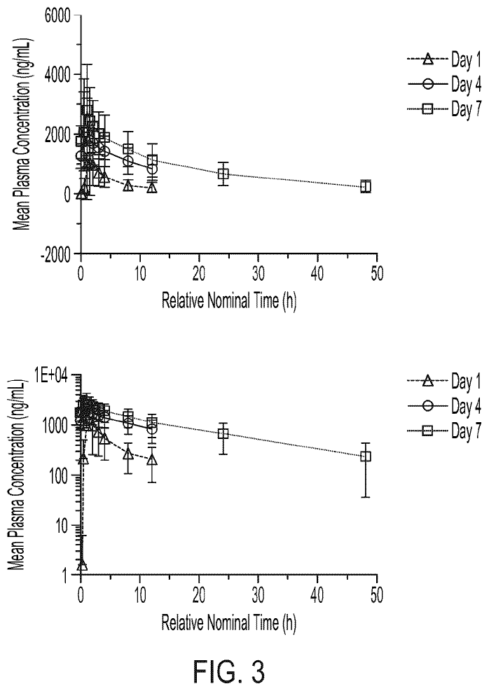

FIG. 3 shows mean vapendavir plasma concentrations vs time profiles (Left=Linear Scale, Right=Log Scale) for Group A (Treatment C) (Days 1-7). Day 1=VPV 1000 mg am and 500 mg pm; Day 4=VPV 500 mg BID; Day 7=VPV 500 mg BID.

Table 5 shows descriptive statistics for PK parameters and discussion (Group A-Treatment C (Days 1 to 7)). Tₘₐₓ is similar between all days. Concentrations obtained, and consequently the exposure observed, are lower following the administration of the loading dose (1000 mg) on Day 1 when compared to the data obtained following the morning dose (500 mg) on Day 4 and Day 7. For Day 7 vs Day 4, a slight accumulation was observed based RA_{AUCtau} and RACₘₐₓ with the geometric LS mean ratio obtained of 132.28 and 126.08%, respectively.

TABLE 5

| | | | | AUC₀₋ₜ | AUC_{0-inf} | AUC₀₋₁₂ | AUC₀₋₂₄ | Cₘₐₓ | Residual |
|---|---|---|---|---|---|---|---|---|---|
| Group | Day | Treatment | Statistics | (h*ng/mL) | (h*ng/mL) | (h*ng/mL) | (h*ng/mL) | (ng/mL) | Area (%) |
| A | 1 | VPV 1000 mg | N | 8 | 6 | 8 | 7 | 8 | 6 |
| | | am and 500 | | | | | | | |
| | | mg pm | | | | | | | |
| | | | Mean | 5372.41 | 8124.70 | 5372.41 | 7308.45 | 1350 | 23.46 |
| | | | CV % | 65.70 | 61.15 | 65.70 | 58.12 | 81.34 | 16.10 |
| | | | Geometric | 4270.89 | 6782.91 | 4270.89 | 6238.29 | 935 | 23.20 |
| | | | Meer | | | | | | |
| | | | Geometric | 90.76 | 78.12 | 90.76 | 69.84 | 134.74 | 16.27 |
| | | | CV % | | | | | | |

| | | | | Tₘₐₓ | T_{%} | K_{ct} | CL/F | Vz/F |
|---|---|---|---|---|---|---|---|---|
| Group | Day | Treatment | Statistics | (h) | (h) | (l/h) | (L/h) | (L) |
| A | 1 | VPV 1000 mg | N | 8 | 6 | 6 | 6 | 6 |
| | | am and 500 | | | | | | |
| | | mg pm | | | | | | |
| | | | Mean | 1.94 | 5.32 | 0.1333 | 180.61 | 1396.50 |
| | | | CV % | 48.65 | 16.98 | 16.55 | 72.70 | 79.85 |
| | | | Geometric | 1.77 | 5.26 | 0.1317 | 147.43 | 1119.01 |
| | | | Meer | | | | | |
| | | | Geometric | 47.13 | 16.92 | 16.92 | 78.12 | 79.07 |
| | | | CV % | | | | | |

| | | | | AUC₀₋ₜ | AUC₀₋ₜₐᵤ | AUC₀₋₂₄ | C_{max ss} | C_{12-trough} |
|---|---|---|---|---|---|---|---|---|
| Group | Day | Treatment | Statistics | (h*ng/mL) | (h*ng/mL) | (h*ng/mL) | (ng/mL) | (ng/mL) |
| A | 4 | VPV 500 mg | N | 8 | 8 | 8 | 8 | 8 |
| | | BID | | | | | | |
| | | | Mean | 15366.24 | 15386.24 | 22546.40 | 2320 | 832 |
| | | | CV % | 41.81 | 41.81 | 46.42 | 55.35 | 49.31 |
| | | | Geometric | 14096.07 | 14095.07 | 20403.07 | 2020 | 747 |
| | | | Mean | | | | | |
| | | | Geometric | 49.18 | 49.18 | 52.54 | 65.35 | 53.77 |
| | | | CV % | | | | | |

| | | | | Tₘₐₓ | T_{%} | K_{ct} |
|---|---|---|---|---|---|---|
| Group | Day | Treatment | Statistics | (h) | (h) | (l/h) |
| A | 4 | VPV 500 mg | N | 8 | 8 | 8 |
| | | BID | | | | |
| | | | Mean | 1.19 | 11.42 | 0.0677 |
| | | | CV % | 74.43 | 32.13 | 40.10 |
| | | | Geometric | 0.96 | 10.86 | 0.0638 |
| | | | Mean | | | |
| | | | Geometric | 74.92 | 36.41 | 36.41 |
| | | | CV % | | | |

| | | | | AUC₀₋ₜ | AUC₀₋ₜₐᵤ | AUC₀₋₂₄ | C_{max ss} | C_{12-trough} |
|---|---|---|---|---|---|---|---|---|
| Group | Day | Treatment | Statistics | (h*ng/mL) | (h*ng/mL) | (h*ng/mL) | (ng/mL) | (ng/mL) |
| A | 7 | VPV 500 mg | N | 7 | 7 | 7 | 7 | 7 |
| | | BID | | | | | | |
| | | | Mean | 41078.11 | 20686.54 | 31186.67 | 2930 | 1120 |
| | | | CV % | 51.80 | 41.96 | 46.13 | 50.68 | 49.41 |
| | | | Geometric | 36457.62 | 19100.44 | 28339.35 | 2590 | 1000 |
| | | | Mean | | | | | |
| | | | Geometric | 57.57 | 46.23 | 51.01 | 60.65 | 55.09 |
| | | | CV % | | | | | |

| | | | | Residual | Tₘₐₓ | T_{%} | K_{ct} |
|---|---|---|---|---|---|---|---|
| Group | Day | Treatment | Statistics | Area (%) | (h) | (h) | (l/h) |
| A | 7 | VPV 500 mg | N | 7 | 7 | 7 | 7 |
| | | BID | | | | | |
| | | | Mean | 9.78 | 1.14 | 14.51 | 0.0506 |
| | | | CV % | 54.10 | 54.84 | 25.11 | 27.11 |
| | | | Geometric | 8.33 | 1.00 | 14.11 | 0.0491 |
| | | | Mean | | | | |
| | | | Geometric | 75.65 | 61.44 | 26.35 | 26.35 |
| | | | CV % | | | | |

| | | | Test | Reference | Ratio¹ |
|---|---|---|---|---|---|
| PK | | Treatment | Geometric | Geometric | Day 7/Day 4 |
| Parameter | Units | Comparison | LSM | LSM | (%) |
| AUCₜₐᵤ | (h*ng/mL) | Day 7/Day 4 | 18645.23 | 14095.07 | 132.28 |
| C_{max ss} | (ng/mL) | Day 7/Day 4 | 2540.79 | 2015.26 | 126.08 |

| | | | 90% | 90% |
|---|---|---|---|---|
| PK | | Treatment | Lower | Upper |
| Parameter | Units | Comparison | CI (%)2 | CI (%)² |
| AUCₜₐᵤ | (h*ng/mL) | Day 7/Day 4 | 110.22 | 158.76 |
| C_{max ss} | (ng/mL) | Day 7/Day 4 | 99.29 | 160.09 |

Table 6 shows descriptive statistics of plasma concentrations vs time for Group B—Treatment C (Days 1 to 7). Measurable concentrations are observed up to 12.0 hours post-dose on Day 1 for all subjects. The first measurable concentration was observed at 0.500 hours for most subjects. Measurable concentrations are observed from 0.00 up to 12.0 hours post-dose on Day 4. On Day 7, measurable concentrations are observed up to 48.0 hours post-dose (end of sampling schedule for Day 7) for all subjects. Based on visual evaluation: Lower concentrations are obtained on Day 1 when compared to Day 4 and 7. Lower concentrations are obtained on Day 4 when compared to Day 7.

TABLE 6

| | | | | Relative Nominal Time (h) | | | | |
|---|---|---|---|---|---|---|---|---|
| | | | | 0.00 | 0.250 | 0.500 | 1.00 | 1.50 |
| Group | Day | Treatment | Statistics | Concentration (ng/mL) | | | | |
| B-1 | 1 | VPV 1000 mg | N | 6 | 6 | 6 | 6 | 6 |
| | | am and 500 | | | | | | |
| | | mg pm | | | | | | |
| | | | Mean | 0.00 | 2.35 | 499 | 1250 | 1930 |
| | | | SD | 0.00 | 5.76 | 495 | 772 | 1300 |
| | | | CV % | NC | 245 | 99.3 | 61.6 | 67.4 |
| | | | Geometric | NC | NC | NC | 1020 | 1560 |
| | | | Mean | | | | | |
| | | | Geometric | NC | NC | NC | 90.1 | 64.7 |
| | | | CV % | | | | | |

| | | | | Relative Nominal Time (h) | | | | |
|---|---|---|---|---|---|---|---|---|
| | | | | 2.00 | 3.00 | 4.00 | 8.00 | 12.0 |
| Group | Day | Treatment | Statistics | Concentration (ng/mL) | | | | |
| B-1 | 1 | VPV 1000 mg | N | 6 | 6 | 6 | 6 | 6 |
| | | am and 500 | Mean | 2150 | 1740 | 1190 | 503 | 426 |
| | | mg pm | SD | 1570 | 1670 | 1080 | 603 | 438 |
| | | | CV % | 73.1 | 95.9 | 90 [symbol] 6 | 120 | 103 |
| | | | Geometric | 1620 | 1220 | 863 | 313 | 305 |
| | | | Mean | | | | | |
| | | | Geometric | 111 | 121 | 110 | 137 | 104 |
| | | | CV % | | | | | |

| | | | | Relative Nominal Time (h) | | | | |
|---|---|---|---|---|---|---|---|---|
| | | | | 0.00 | 0.250 | 0.500 | 1.00 | 1.50 |
| Group | Day | Treatment | Statistics | Concentration (ng/mL) | | | | |
| B-1 | 4 | VPV 600 mg | N | 6 | 6 | 6 | 6 | 6 |
| | | BID | | | | | | |
| | | | Mean | 1670 | 1630 | 2430 | 2920 | 2680 |
| | | | SD | 1260 | 1240 | 1310 | 1180 | 1220 |
| | | | CV % | 75.4 | 76 [symbol] 2 | 53.7 | 40.5 | 45.6 |
| | | | Geometric | 1410 | 1370 | 2170 | 2700 | 2450 |
| | | | Mean | | | | | |
| | | | Geometric | 63.0 | 63.8 | 55.3 | 47.7 | 50.1 |
| | | | CV % | | | | | |
| | | | | Relative Nominal Time (h) | | | | |
| | | | | 2.00 | 3.00 | 4.00 | 8.00 | 12.0 |
| Group | Day | Treatment | Statistics | Concentration (ng/mL) | | | | |
| B-1 | 4 | VPV 600 mg | N | 6 | 6 | 6 | 6 | 6 |
| | | BID | | | | | | |
| | | | Mean | 2460 | 2270 | 1860 | 1550 | 1190 |
| | | | SD | 1220 | 1280 | 1090 | 971 | 794 |
| | | | CV % | 49.8 | 56.4 | 58.8 | 62.8 | 66.6 |
| | | | Geometric | 2250 | 2030 | 1660 | 1370 | 1040 |
| | | | Mean | | | | | |
| | | | Geometric | 47.7 | 54.3 | 52.1 | 54.2 | 59.2 |
| | | | CV % | | | | | |

| | | | | Relative Nominal Time (h) | | | | | |
|---|---|---|---|---|---|---|---|---|---|
| | | | | 0.00 | 0.250 | 0.500 | 1.00 | 1.50 | 2.00 |
| Group | Day | Treatment | Statistics | Concentration (ng/mL) | | | | | |
| B-1 | 7 | VPV 500 mg | N | 6 | 6 | 6 | 6 | 6 | 6 |
| | | BID | Mean | 1560 | 1510 | 1930 | 2800 | 2830 | 2680 |
| | | | SD | 665 | 687 | 499 | 1160 | 849 | 953 |
| | | | CV % | 42.5 | 45.6 | 25.8 | 41.4 | 30.0 | 35.5 |
| | | | Geometric | 1470 | 1400 | 1880 | 2030 | 2730 | 2540 |
| | | | Mean | | | | | | |
| | | | Geometric | 38.7 | 41.4 | 24.2 | 38.4 | 29.7 | 37.8 |
| | | | CV % | | | | | | |

| | | | | Relative Nominal Time (h) | | | | | |
|---|---|---|---|---|---|---|---|---|---|
| | | | | 3.00 | 4.00 | 8.00 | 12.0 | 24.0 | 48.0 |
| Group | Day | Treatment | Statistics | Concentration (ng/mL) | | | | | |
| B-1 | 7 | VPV 500 mg | N | 6 | 6 | 6 | 6 | 6 | 6 |
| | | BID | Mean | 2160 | 1830 | 1450 | 1040 | 760 | 285 |
| | | | SD | 810 | 740 | 604 | 514 | 506 | 240 |
| | | | CV % | 37.5 | 40 [symbol] 5 | 41.6 | 49.4 | 66.7 | 84.4 |
| | | | Geometric | 2050 | 1730 | 1370 | 952 | 661 | 223 |
| | | | Mean | | | | | | |
| | | | Geometric | 33.0 | 36.0 | 37.7 | 47.3 | 57.9 | 84.8 |
| | | | CV % | | | | | | |

[symbol]

indicates data missing or illegible when filed

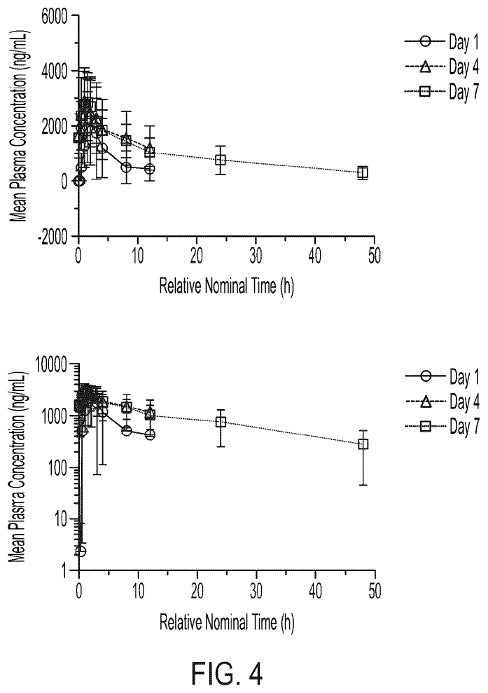

FIG. 4 depicts mean vapendavir plasma concentrations vs time profiles (Left=Linear Scale, Right=Log Scale) for Group B—Treatment C (Days 1-7). SD=Standard deviation
Day 1=VPV 1000 mg am and 500 mg pm; Day 4=VPV 500 mg BID; Day 7=VPV 500 mg BID.

Table 7 shows descriptive statistics for PK parameters for Group B—Treatment C (Days 1 to 7). Tₘₐₓ is similar between all days. Concentrations obtained, and consequently the exposure observed, are lower following the administration of the loading dose (1000 mg) on Day 1 when compared to the data obtained following the morning dose (500 mg) on Day 4 and Day 7. For Day 7 vs Day 4, no accumulation was observed based RA_{AUCtau} and RACₘₐₓ with the geometric LS mean ratio obtained of 100.57 and 95.38%, respectively.

TABLE 7

| | | | | AUC₀₋ₜ | AUC_{0-inf} | AUC₀₋₁₂ | AUC₀₋₂₄ | Cₘₐₓ | Residual |
|---|---|---|---|---|---|---|---|---|---|
| Group | Day | Treatment | Statistics | (h*ng/mL) | (h*ng/mL) | (h*ng/mL) | (h*ng/mL) | (ng/mL) | Area (%) |
| B-1 | 1 | VPV 1000 | N | 6 | 5 | 6 | 6 | 6 | 5 |
| | | mg am | | | | | | | |
| | | and 500 | | | | | | | |
| | | mg pm | | | | | | | |
| | | | Mean | 10667.31 | 14669.55 | 10667.31 | 13078 [symbol] 26 | 2340 | 22.78 |
| | | | CV % | 34.10 | 97.29 | 84.10 | 90.40 | 62.35 | 24.25 |
| | | | Geometric | 8296.85 | 10582.42 | 8296.85 | 9954.27 | 2000 | 22.26 |
| | | | Mean | | | | | | |
| | | | Geometric | 39.67 | 109.29 | 89.67 | 92.53 | 68.66 | 24.17 |
| | | | CV % | | | | | | |

| | | | | Tₘₐₓ | T_{%} | K_{ct} | CL/F | Vz/F |
|---|---|---|---|---|---|---|---|---|
| Group | Day | Treatment | Statistics | (h) | (h) | (l/h) | (L/h) | (L) |
| B-1 | 1 | VPV 1000 | N | 6 | 5 | 5 | 5 | 5 |
| | | mg am | | | | | | |
| | | and 500 | | | | | | |
| | | mg pm | | | | | | |
| | | | Mean | 1.75 | 5.38 | 0.1356 | 125.04 | 958.86 |
| | | | CV % | 43.33 | 25.47 | 24.53 | 80.15 | 80.75 |
| | | | Geometric | 1.62 | 5.24 | 0.1323 | 94.5 | 714.44 |
| | | | Mean | | | | | |
| | | | Geometric | 45.48 | 25.51 | 25.51 | 109.29 | 109.66 |
| | | | CV % | | | | | |

| | | | | AUC₀₋ₜ | AUCₜₐᵤ | AUC₀₋₂₄ | C_{max ss} | C_{12-trough} | Residual |
|---|---|---|---|---|---|---|---|---|---|
| Group | Day | Treatment | Statistics | (h*ng/mL) | (h*ng/mL) | (h*ng/mL) | (ng/mL) | (ng/mL) | Area (%) |
| B-1 | 4 | VPV500 mg | N | 6 | 6 | 6 | 6 | 6 | 5 |
| | | BID | | | | | | | |
| | | | Mean | 21581.16 | 21581.16 | 32055.86 | 3280 | 1190 | 46.82 |
| | | | CV % | 55.46 | 55.46 | 61.28 | 32.77 | 66.60 | 22.47 |
| | | | Geometric | 19521.95 | 19521.95 | 28401.17 | 3130 | 1040 | 45.81 |
| | | | Mean | | | | | | |
| | | | Geometric | 49.10 | 49.10 | 54.36 | 35.67 | 59.21 | 23.31 |
| | | | CV % | | | | | | |

| | | | | Tₘₐₓ | T_{%} | K_{ct} |
|---|---|---|---|---|---|---|
| Group | Day | Treatment | Statistics | (h) | (h) | (l/h) |
| B-1 | 4 | VPV 500 mg | N | 6 | 6 | 6 |
| | | BID | | | | |
| | | | Mean | 1.42 | 11.96 | 0.0631 |
| | | | CV % | 64.76 | 32.84 | 30.14 |
| | | | Geometric | 1.20 | 11.45 | 0.0605 |
| | | | Mean | | | |
| | | | Geometric | 69.42 | 32.87 | 32.87 |
| | | | CV % | | | |

| | | | | AUC₀₋ₜ | AUC₀₋ₜₐᵤ | AUC₀₋₂₄ | C_{max ss} | C_{12-trough} | Residual |
|---|---|---|---|---|---|---|---|---|---|
| Group | Day | Treatment | Statistics | (h*ng/mL) | (h*ng/mL) | (h*ng/mL) | (ng/mL) | (ng/mL) | Area (%) |
| B-1 | 7 | VPV 500 mg | N | 6 | 6 | 6 | 6 | 6 | 6 |
| | | BID | | | | | | | |
| | | | Mean | 42910.74 | 20633.50 | 31323.24 | 3160 | 1040 | 12.87 |
| | | | CV % | 51.27 | 36.66 | 43.22 | 36.74 | 49.35 | 44.23 |
| | | | Geometric | 39327.60 | 19634.14 | 29359.00 | 2960 | 952 | 11.75 |
| | | | Mean | | | | | | |
| | | | Geometric | 45.22 | 34.36 | 39.00 | 37.92 | 47.29 | 51.82 |
| | | | CV % | | | | | | |

| | | | | Tₘₐₓ | T_{%} | K_{ct} |
|---|---|---|---|---|---|---|
| Group | Day | Treatment | Statistics | (h) | (h) | (l/h) |
| B-1 | 7 | VPV 500 mg | N | 6 | 6 | 6 |
| | | BID | | | | |
| | | | Mean | 1.50 | 16.76 | 0.0427 |
| | | | CV % | 29.61 | 20.11 | 19.45 |
| | | | Geometric | 1.44 | 16.49 | 0 [symbol] 0420 |
| | | | Mean | | | |
| | | | Geometric | 31.92 | 19.94 | 19.94 |
| | | | CV % | | | |

| | | | Reference | Test | Ratio¹ |
|---|---|---|---|---|---|
| PK | | Treatment | Geometric | Geometric | Day 7/Day 4 |
| Parameter | Units | Comparison | LSM | LSM | (%) |
| AUCₜₐᵤ | (h*ng/mL) | Day 7/Day 4 | 19521.95 | 19634.14 | 100.57 |
| C_{max ss} | (ng/mL) | Day 7/Day 4 | 3129.50 | 2984.94 | 95.38 |

| | | | 90% | 90% |
|---|---|---|---|---|
| PK | | Treatment | Lower | Upper |
| Parameter | Units | Comparison | CI (%)² | CI (%)² |
| AUCₜₐᵤ | (h*ng/mL) | Day 7/Day 4 | 83.84 | 120.65 |
| C_{max ss} | (ng/mL) | Day 7/Day 4 | 77.61 | 117.21 |

[symbol]

indicates data missing or illegible when filed

Table 8 shows a comparison between normal healthy volunteers and participants with a diagnosis of COPD (Part A vs. Party B).

| | | | | | | | 90% | 90% |
|---|---|---|---|---|---|---|---|---|
| | | | | Test | Reference | Ratio¹ | Lower | Upper |
| | PK | | Treatment | Geometric | Geometric | HVS/COPD | CI | CI |
| Day | Parameter | Units | Comparisons | LSM | LSM | (%) | (%)² | (%)² |
| 1 | AUC₀₋₁₂ | h*ng/mL | HVS/COPD | 4270.89 | 8296.85 | 51.48 | 20.18 | 108.65 |
| | Cₘₐₓ | ng/mL | HVS/COPD | 935.19 | 1999.26 | 46.82 | 24.48 | 108.25 |
| 4 | AUCₜₐᵤ | h*ng/mL | HVS/COPD | 19521.95 | 14095.07 | 72.20 | 46.14 | 112.98 |
| | C_{max ss} | ng/mL | HVS/COPD | 3129.50 | 2015.26 | 64.40 | 39.52 | 104.94 |
| 7 | AUCₜₐᵤ | h*ng/mL | HVS/COPD | 19634.14 | 19100.44 | 97.28 | 65.53 | 144.43 |
| | C_{max ss} | ng/mL | HVS/COPD | 2984.94 | 2585.45 | 86.62 | 53.54 | 140.14 |

Day 1: A higher exposure was observed in participants with a diagnosis of COPD when compared to normal healthy volunteers. Indeed, the geometric LS mean ratio (normal healthy volunteers vs. participants with a diagnosis of COPD) obtained are 51.48% and 46.82%.

Day 4: A higher exposure was observed in participants with a diagnosis of COPD when compared to normal healthy volunteers. Indeed, the geometric LS mean ratio (HVS vs COPD) obtained are 72.20% and 64.40%.

For Day 7: A comparable exposure was observed in participants with a diagnosis of COPD when compared to normal healthy volunteers. Indeed, the geometric LS mean ratio and confidence interval ratios obtained are 97.28% (65.53% to 144.43%) and 86.62% (53.54% to 140.14%). However, confidence interval ratios obtained are wide demonstrating the high variability of the data obtained.

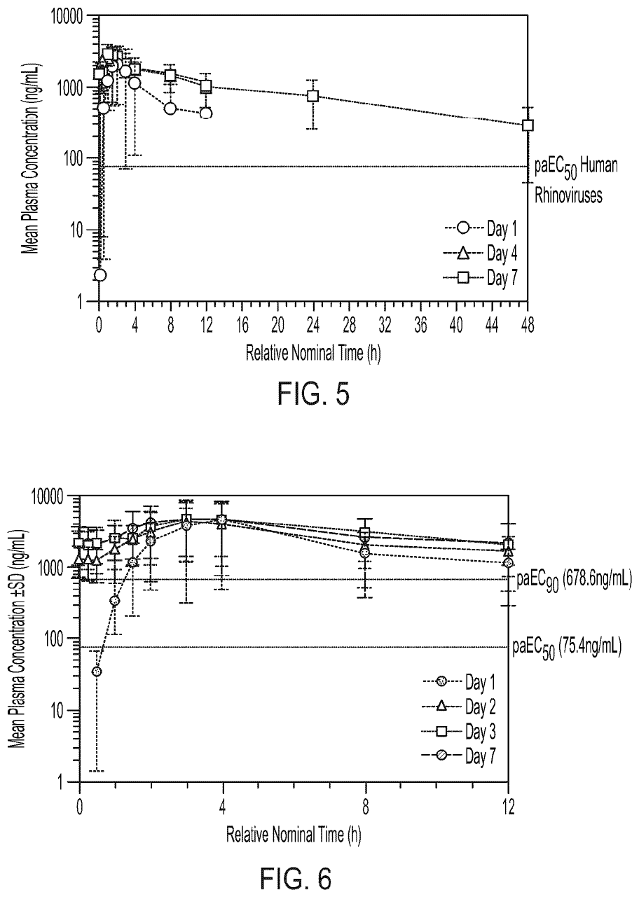

In participants with a diagnosis of COPD (Group B), administration of vapendavir for 7 days ((Day 1=VPV 1000 mg am and 500 mg pm: Day 4=VPV 500 mg BID: Day 7=VPV 500 mg BID)) results in a mean plasma concentration that exceeds the protein adjusted EC₅₀ for human rhinoviruses. See FIG. 5. This data demonstrates that the treatment regimen disclosed herein results in mean plasma concentration levels that exceed the protein adjusted EC₅₀ for human rhinoviruses suggesting it would be therapeutically effective.

Administration of 500 mg vapendavir in normal healthy volunteers that were in a fed state prior to administration results in a Cₘₐₓ of 2060 ng/ml (CV % 5 ng/ml) compared to 687 ng/ml (CV % 93 ng/ml) in normal healthy volunteers that were in fasted state prior to administration.

Surprisingly, administration of a 1000 mg loading dose in COPD resulted in a Cₘₐₓ of 2150 ng/ml (CV % 73 ng/ml) compared to a Cₘₐₓ of 1060 ng/ml (CV % 106 ng/ml) in normal healthy volunteers suggesting that use of a loading dose of 1000 mg in participants with a diagnosis of COPD results in a higher plasma concentration and has a greater effect than in normal healthy volunteers. This is surprising, at least in part because COPD patients tend to be advanced in age (typically over the age of 70 years old) and are known to have decreased drug absorption.

This data suggests that use of a loading dose could have significant benefits for the treatment of COPD patients and in addition, treatment of patients that are in a fed state can further increase vapendavir levels compared to treatment of patients that are in a fasted state.

### Example 3—Supplementary Pharmacokinetic Results from Participants with a Diagnosis of COPD

This study was conducted to determine the food effect on vapendavir (VPV) exposure in participants with COPD including to determine the overall VPV exposure in participants with COPD, fed (loading dose followed by 7-day maintenance dose), and to determine the variation in VPV exposure in participants with COPD, fed.

Six new participants with COPD (no cross over from previous study described in Example 2) were dosed with a 1,000 mg loading dose followed by 500 mg BID for a full 7-day course. Dosing occurred fed, with moderate fat diet, not high fat diet to better approximate actual diet of participants in their homes for the challenge study and P2/3 RCTs. PK sampling was performed on Days 2 and 3, removed Day 4; and PK sampling extended to 144 hours post-final dose on Day 7 to better characterize terminal elimination half-life.

Four participants experienced a total of eight adverse events (AEs): itching, lightheadedness, rash, increased urinary frequency (intermittent), abrasion, daytime sleepiness, bloating, and neck pain. All AEs were Grade 1 (mild) and were unrelated except for daytime sleepiness that was assessed by the investigator as possibly related.

In participants with COPD dosed with a 1,000 mg loading dose followed by 500 mg BID for a full 7-day course, administration results in a mean plasma concentration that exceeds the protein adjusted EC₅₀ for human rhinoviruses. See FIG. 6 which shows VPV Concentrations over Time. This data demonstrates that the treatment regimen disclosed herein results in mean plasma concentration levels that exceed the protein adjusted EC₅₀ and EC₉₀ for human rhinoviruses suggesting it would be therapeutically effective.

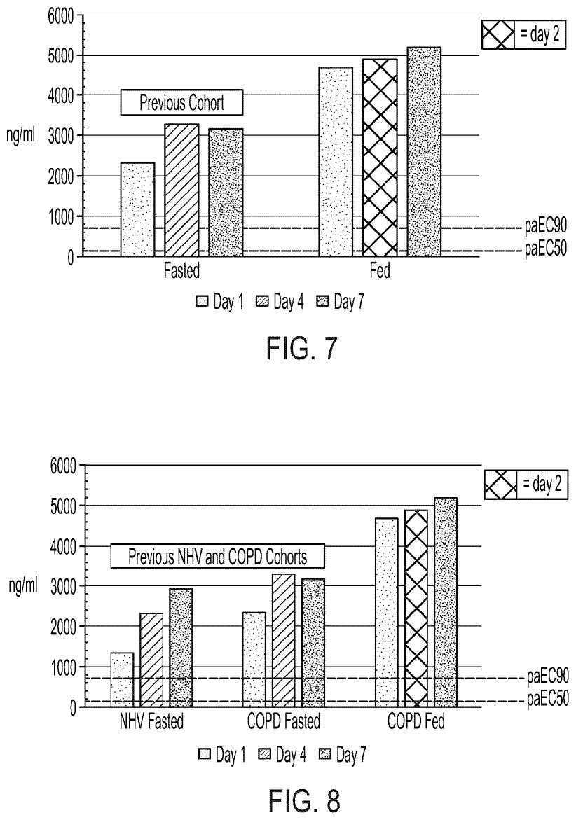

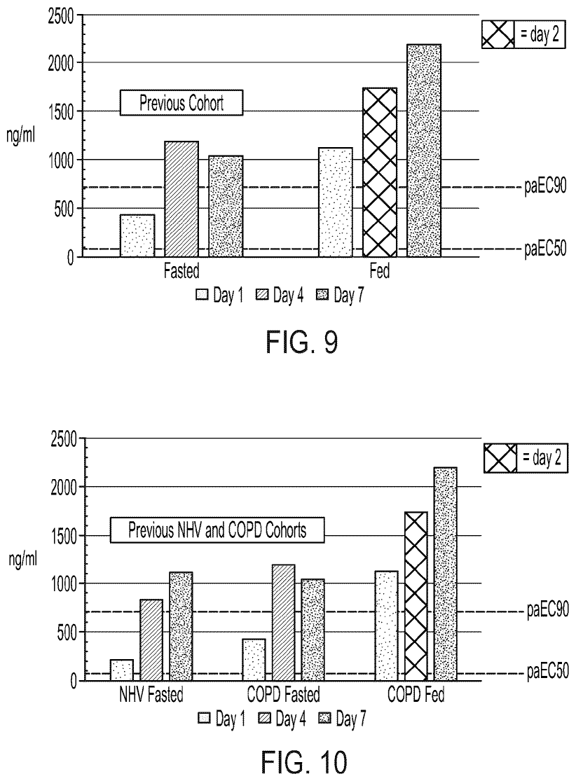

Administering VPV to participants that are fed results in increased Cₘₐₓ concentrations compared with participants that were fasted. See FIG. 7 which shows the mean Cmax in fasted versus fed participants with COPD after 1, 4 and 7 days for the fasted group (Group B—Treatment C (Days 1-7 (Day 1=VPV 1000 mg am and 500 mg pm: Day 4=VPV 500 mg BID: Day 7=VPV 500 mg BID)) and 1, 2, and 7 days for the fed group (1000 mg loading dose followed by 500 mg BID for a full 7-day course). FIG. 8 shows the mean Cₘₐₓ in fasted versus fed participants with COPD as well as normal healthy volunteers after 1, 4 and 7 days for the fasted group (Group A (Normal healthy volunteers) and Group B (COPD)—Treatment C (Days 1-7 (Day 1=VPV 1000 mg am and 500 mg pm: Day 4=VPV 500 mg BID: Day 7=VPV 500 mg BID)) and 1, 2, and 7 days for the fed group (1000 mg loading dose followed by 500 mg BID for a full 7-day course). FIG. 9 shows the mean Cₘᵢₙ (12 hours) in fasted versus fed participants with COPD after 1, 4 and 7 days for the fasted group (Group B—Treatment C (Days 1-7 (Day 1=VPV 1000 mg am and 500 mg pm: Day 4=VPV 500 mg BID: Day 7=VPV 500 mg BID)) and 1, 2, and 7 days for the fed group (1000 mg loading dose followed by 500 mg BID for a full 7-day course). FIG. 10 shows the mean Cₘᵢₙ (12 hours) in fasted versus fed participants with COPD as well as normal healthy volunteers after 1, 4 and 7 days for the fasted group (Group A (Normal healthy volunteers) and Group B (COPD)—Treatment C (Days 1-7 (Day 1=VPV 1000 mg am and 500 mg pm: Day 4=VPV 500 mg BID: Day 7=VPV 500 mg BID)) and 1, 2, and 7 days for the fed group (1000 mg loading dose followed by 500 mg BID for a full 7-day course).

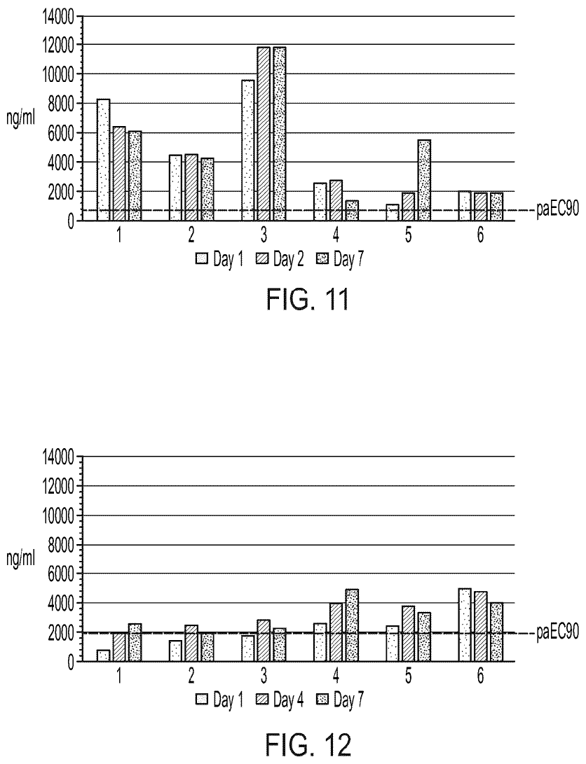

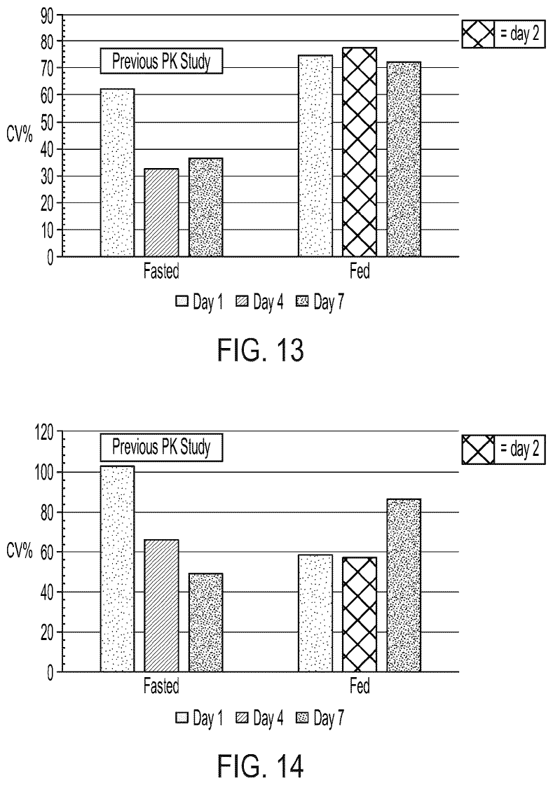

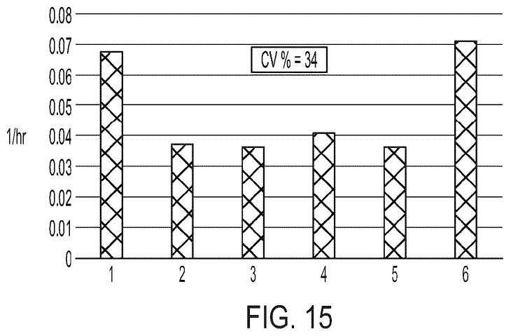

FIG. 11 shows Cₘₐₓ for each day of treatment for each of the COPD subjects in the study where each subject was fed. FIG. 12 shows Cₘₐₓ for each day of treatment for each of the COPD subjects in the study where each subject was fasted (Group B). FIG. 13, shows the coefficient of variation for the Cₘₐₓ in fasted versus fed participants with COPD after 1, 4 and 7 days for the fasted group (Group B—Treatment C (Days 1-7 (Day 1=VPV 1000 mg am and 500 mg pm: Day 4=VPV 500 mg BID: Day 7=VPV 500 mg BID)) and 1, 2, and 7 days for the fed group (1000 mg loading dose followed by 500 mg BID for a full 7-day course). FIG. 14 shows the coefficient of variation for the Cₘᵢₙ in fasted versus fed participants with COPD after 1, 4 and 7 days for the fasted group (Group B—Treatment C (Days 1-7 (Day 1=VPV 1000 mg am and 500 mg pm: Day 4=VPV 500 mg BID: Day 7=VPV 500 mg BID)) and 1, 2, and 7 days for the fed group (1000 mg loading dose followed by 500 mg BID for a full 7-day course). FIG. 15 depicts Kₑₗ (Elimination Rate Constant) for each of the 6 Participants for the fed group (1000 mg loading dose followed by 500 mg BID for a full 7-day course) at day 7.

This data suggests that use of a loading dose could have significant benefits for the treatment of COPD patients and in addition, treatment of patients that are in a fed state can further increase vapendavir levels compared to treatment of patients that are in a fasted state.

### Example 4—Exemplary Dosing Regimens for 1000 mg Loading Dose and Thirteen Doses of 500 mg Every 12 Hours

| 1000 mg loading dose administered in the | 1000 mg loading dose administered in the |
|---|---|
| morning on Day 1 | afternoon or evening on Day 1 |
| First maintenance dose of 500 mg | First maintenance dose of 500 mg |
| administered in the evening on Day 1 | administered in the morning on Day 2 |
| Second maintenance dose of 500 mg | Second maintenance dose of 500 mg |
| administered in the morning on Day 2 | administered in the evening on Day 2 |
| Third maintenance dose of 500 mg | Third maintenance dose of 500 mg |
| administered in the evening on Day 2 | administered in the morning of Day 3 |
| Fourth maintenance dose of 500 mg | Fourth maintenance dose of 500 mg |
| administered in the morning on Day 3 | administered in the evening on Day 3 |
| Fifth maintenance dose of 500 mg | Fifth maintenance dose of 500 mg |
| administered in the evening on Day 3 | administered in the morning on Day 4 |
| Sixth maintenance dose of 500 mg | Sixth maintenance dose of 500 mg |
| administered in the morning on Day 4 | administered in the evening on Day 4 |
| Seventh maintenance dose of 500 mg | Seventh maintenance dose of 500 mg |
| administered in the evening on Day 4 | administered in the morning on Day 5 |
| Eighth maintenance dose of 500 mg | Eighth maintenance dose of 500 mg |
| administered in the morning on Day 5 | administered in the evening on Day 5 |
| Ninth maintenance dose of 500 mg | Ninth maintenance dose of 500 mg |
| administered in the evening on Day 5 | administered in the morning on Day 6 |
| Tenth maintenance dose of 500 mg | Tenth maintenance dose of 500 mg |
| administered in the morning on Day 6 | administered in the evening on Day 6 |
| Eleventh maintenance dose of 500 mg | Eleventh maintenance dose of 500 mg |
| administered in the evening on Day 6 | administered in the morning on Day 7 |
| Twelfth maintenance dose of 500 mg | twelfth maintenance dose of 500 mg |
| administered in the morning on Day 7 | administered in the evening on Day 7 |
| Thirteenth maintenance dose of 500 mg | Thirteenth maintenance dose of 500 mg |
| administered in the evening on Day 7 | administered in the morning on Day 8 |

This disclosure is not limited to the particular systems, devices and methods described, as these may vary. The terminology used in the description is for the purpose of describing the particular versions or embodiments only and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations.” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example. “a system having at least one of A, B, and C” would include but not be limited to systems that have A alone. B alone. C alone. A and B together. A and C together. B and C together, and/or A, B, and C together, et cetera). In those instances where a convention analogous to “at least one of A, B, or C, et cetera” is used, in general such a construction is intended in the sense one having skill in the art would understand the convention (for example. “a system having at least one of A, B, or C” would include but not be limited to systems that have A alone. B alone. C alone. A and B together. A and C together. B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to.” “at least.” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 cells refers to groups having 1, 2, 3, 4, or 5 compounds, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

What is claimed is:

1. A method of treating a respiratory enterovirus infection in a human subject in need thereof, comprising orally administering
to the subject a therapeutically effective amount of vapendavir or a pharmaceutically acceptable salt thereof, thereby treating the respiratory enterovirus infection in the subject.

2. The method of claim 1, wherein the therapeutically effective amount of vapendavir or a pharmaceutically acceptable salt thereof is between about 250 mg and about 2,000 mg per day.

3. The method of claim 1, wherein the respiratory enterovirus is selected from a rhinovirus, echovirus, EV-68, EV-71, coxsackie virus, a non-polio enterovirus and combinations thereof.

4. The method of claim 1, wherein the therapeutically effective amount of vapendavir or a pharmaceutically acceptable salt thereof is a single loading dose of about 1,000 mg followed by a maintenance dose of about 500 mg every 12 hours for about 4 to about 10 days.

5. The method of claim 4, wherein the therapeutically effective amount of vapendavir or a pharmaceutically acceptable salt thereof is a single loading dose of about 1,000 mg per followed a maintenance dose of about 500 mg every 12 hours for a total of about 13 maintenance doses.

6. The method of claim 1, wherein the therapeutically effective amount of vapendavir or a pharmaceutically acceptable salt thereof is an amount that achieves a Cmax of about 2000 to about 12000 ng/ml.

7. The method of claim 1, wherein the subject is in a fed state.

8. The method of claim 4, wherein the single loading dose and maintenance dose are administered within about 30 minutes of the subject consuming a meal with solid food.

9. The method of claim 8, wherein administering the single loading dose and maintenance dose to a subject within about 30 minutes of the subject consuming a meal with solid food results in a higher plasma concentration of vapendavir or a pharmaceutically acceptable salt thereof than administering the single loading dose and maintenance dose to a subject in a fasted state.

10. The method of claim 1, wherein the subject has an underlying condition selected from chronic obstructive pulmonary diseases (COPD), asthma, mixed COPD and asthma; cystic fibrosis (CF); bronchiolitis obliterans; fibrosis such as tuberculosis (TB), idiopathic pulmonary fibrosis (IPF), other major respiratory diagnosis (e.g., pneumonia, aspergillosis), non-CF bronchiectasis, COPD due to alpha-1 antitrypsin deficiency, active allergic rhinitis, nasal disease, (e.g. nasal polyposis, significant septal deviation, chronic rhinosinusitis, etc.); primary ciliary dyskinesia; bronchitis; pulmonary hypertension; pulmonary edema; acute respiratory distress syndrome (ARDS); pneumoconiosis; interstitial lung disease; mesothelioma; sickle cell disease, cancer, or any combination thereof.

11. The method of claim 1, wherein the subject has COPD and has been diagnosed with GOLD stage 1 COPD, GOLD stage 2 COPD, GOLD stage 3 COPD, or GOLD stage 4 COPD.

12. The method of claim 14, wherein the subject is receiving stable maintenance therapy for COPD.

13. The method of claim 1, wherein treating the respiratory enterovirus infection in the subject comprises decreasing a viral load in the sputum, nasal passages, or a combination thereof in the subject compared with a viral load in the sputum, nasal passages, or a combination thereof of the subject prior to treatment.

14. The method of claim 1, wherein treating the respiratory enterovirus infection in the subject comprises decreasing the subjects' Evaluating Respiratory Symptoms in COPD Score (E-RS) compared to the subjects' Evaluating Respiratory Symptoms in COPD Score (E-RS) prior to treatment.

15. The method of claim 1, wherein treating the respiratory enterovirus infection in the subject comprises preventing an increase in the subjects' Evaluating Respiratory Symptoms in COPD Score (E-RS) compared to the subjects' Evaluating Respiratory Symptoms in COPD Score (E-RS) score prior to treatment.

16. The method of claim 1, wherein treating the respiratory enterovirus infection in the subject comprises decreasing the subjects' peak lower respiratory symptom score (LRSS) compared to the subjects' peak lower respiratory symptom score (LRSS) prior to treatment.

17. The method of claim 1, wherein treating the respiratory enterovirus infection in the subject comprises preventing an increase in the subjects' peak lower respiratory symptom score (LRSS) compared to the subjects' peak lower respiratory symptom score (LRSS) prior to treatment.

18. The method of claim 1, wherein treating the respiratory enterovirus infection in the subject comprises decreasing the subjects' peak upper respiratory symptom score (URSS) compared to the subjects' peak upper respiratory symptom score (URSS) prior to treatment.

19. The method of claim 1, wherein treating the respiratory enterovirus infection in the subject comprises preventing an increase in the subjects' peak upper respiratory symptom score (URSS) compared to the subjects' peak upper respiratory symptom score (URSS) prior to treatment.

20. The method of claim 1, wherein treating the respiratory enterovirus infection in the subject comprises decreasing or preventing an increase in the subjects' EXAcerbation of Chronic Pulmonary Disease Tool-Patient Reported Outcome (EXACT-PRO) compared to the subjects' EXAcerbation of Chronic Pulmonary Disease Tool-Patient Reported Outcome (EXACT-PRO) prior to treatment.

21. The method of claim 1, wherein treating the respiratory enterovirus infection in the subject comprises decreasing or preventing an increase in the subjects' EXAcerbation of Chronic Pulmonary Disease Tool-Patient Respiratory Symptoms (EXACT-RS) compared to the subjects' EXAcerbation of Chronic Pulmonary Disease Tool-Patient Respiratory Symptoms (EXACT-RS) prior to treatment.

22. The method of claim 1, wherein treating the respiratory enterovirus infection in the subject comprises preventing a decrease in the subjects' lung function compared to the subjects' lung function prior to treatment.

23. The method of claim 22, wherein the subjects' lung function is measured by Forced Expiratory Volume-1 (FEV₋₁; in liters and % predicted), Forced Vital Capacity (FVC; in liters and % predicted) FEV₁/FVC ratio and peak expiratory flow (PEF) or any combination thereof.

24. The method of claim 1, wherein treating the respiratory enterovirus infection in the subject comprises reducing or preventing an increase in the subjects' peak nasal lavage viral load compared to the subjects' peak nasal lavage viral load prior to treatment.

25. The method of claim 1, wherein treating the respiratory enterovirus infection in the subject comprises reducing or preventing an increase in the subjects' peak sputum lavage viral load compared to the subjects' peak sputum lavage viral load prior to treatment.

26. The method of claim 1, wherein treating the respiratory enterovirus infection in the subject comprises reducing or preventing an increase in the subjects' AUC nasal viral load compared to the subjects' AUC nasal viral load prior to treatment.

27. The method of claim 1, wherein treating the respiratory enterovirus infection in the subject comprises reducing or preventing an increase in the subjects' AUC sputum viral load compared to the subjects' AUC sputum viral load prior to treatment.

28. The method of claim 1, wherein treating the respiratory enterovirus infection in the subject comprises reducing the duration of viral shedding.

29. The method of claim 1, wherein treating the respiratory enterovirus infection in the subject comprises reducing or preventing an increase in the subjects' peak sputum bacterial load compared to the subjects' peak sputum lavage bacterial load prior to treatment.

30. The method of claim 1, wherein treating the respiratory enterovirus infection in the subject comprises reducing or preventing an increase in the subjects' AUC sputum bacterial load compared to the subjects' AUC sputum bacterial load prior to treatment.

31. The method of claim 1, wherein treating the respiratory enterovirus infection in the subject comprises reducing the number of days wherein the subject is positive for bacteria in the sputum.
